(19) United States
(12) Patent Application Publication
(10) Pub. No.: US 2013/0035255 A1
(43) Pub. Date: Feb. 7, 2013

(54) CONTROLLED RELEASE HYBRID SYSTEMS

(71) Applicant: Tanya Sandrock, Salt Lake City, UT (US)

(72) Inventor: Tanya Sandrock, Salt Lake City, UT (US)

(73) Assignee: INTEGRATECH PROTEOMICS, LLC, Salt Lake City, UT (US)

(21) Appl. No.: 13/636,024

(22) PCT Filed: Mar. 25, 2011
(86) PCT No.: PCT/US11/29992

§ 371 Date: Sep. 19, 2012

Related U.S. Application Data
(60) Provisional application No. 61/318,193, filed on Mar. 26, 2010.

(51) Int. Cl. G01N 33/53; C40B 30/04

(52) U.S. Cl. 506/9; 435/6.13

(57) ABSTRACT

Described herein are controlled release hybrid systems and methods of use thereof. In certain embodiments, the controlled release hybrid systems can be used to screen for interactions between candidate molecules. In addition, these controlled release hybrid systems can be modified to screen for chemical inhibitors of interacting candidate molecules. In another embodiment, the controlled release hybrid systems described herein can be modified to screen for mutant molecules (e.g., peptides, proteins, polypeptides, portions of proteins, portions of polypeptides, or any combination thereof) when compared to candidate molecules that were previously shown to interact. Upon identifying these mutant molecules, the controlled release hybrid systems can be further utilized to identify peptide, chemicals, or a combination thereof that potentially act as inhibitors of these mutant molecules. In this embodiment, the controlled release systems can be readily modified for personalized medication applications.

### STATEMENT REGARDING FEDERALLY SPONSORED RESEARCH OR DEVELOPMENT

This invention was made with Government Support under Agreement 1R43AI084610 awarded to Tanya Sandrock by the National Institutes of Health. The Government has certain rights in the invention.

### FIELD OF THE INVENTION

The invention is generally related to the field of hybrid systems (e.g., yeast hybrid systems including one hybrid, two hybrid, and three hybrid systems), more particularly to methods and compositions for controlled release hybrid systems.

### BACKGROUND OF THE INVENTION

Over the past few decades, there has been much interest in identifying and evaluating protein-protein interactions. Numerous biochemical approaches, which include yeast hybrid assays (e.g., yeast one-hybrid, two-hybrid, and three hybrid assays) and immunoprecipitation assays (e.g., pull-down assays), have been designed to identify interacting proteins. These biochemical approaches generally employ the affinities between interacting proteins to isolate proteins in a bound state.

In general, yeast two-hybrid systems utilize the expression of chimeric genes and detect protein-protein interactions via the activation of reporter-gene expression. Reporter-gene expression occurs after reconstitution of a functional transcription factor caused by the association of fusion proteins encoded by the chimeric genes. More specifically, yeast two-hybrid systems typically utilize chimeric gene expression of (1) a construct having at least (a) one DNA-binding domain (DBD) of a transcriptional activator and (b) at least one test protein domain (“bait”) and expression of (2) a second construct having at least (a) one activation domain (AD) of a transcriptional activator and (b) at least a second test protein domain (“prey”). Once expressed, if the bait and prey test proteins interact with one another, the transcriptional activator domains (i.e., the DBD and the AD) are brought together reconstituting the transcriptional activator and ultimately activating gene expression of a reporter gene controlled by the transcriptional activator. Therefore, reporter-gene expression confirms interaction between the bait and prey proteins. See, e.g., U.S. Pat. No. 5,283,173. To date, yeast two-hybrid systems have proven to be a powerful tool for the discovery of specific protein interactions in vivo. It is also important to note that these techniques are not limited to yeast-based systems but can be readily adapted for use in various prokaryotic and eukaryotic systems (e.g., various fungal strains, arthropods, plants, plant cells, and mammalian cells).

In addition to the prototypical yeast two-hybrid system described above, this technique can be modified in numerous ways. For example, two-hybrid systems can include the additional feature of positive selection. With positive selection, the interaction of bait and prey results in survival of the cell. Cells containing proteins that either do not interact strongly or do not interact at all fail to grow in selection, and these proteins are no longer considered as candidates for potential protein-protein interactions. Therefore, positive selection eliminates cells containing irrelevant bait and prey pairs without intervention from the scientist or other automated analysis. While positive selection often proves advantageous in the context of two-hybrid systems, the current methods of selection are limited to an “all or nothing” auxotrophic nutrient, antibiotic selection or other means of affecting survival (Fields and Song, (1989) Nature 340, 245-246; Gyuris et al. (1993) Cell 75, 791-803; 1993; Bai and Elledge, (1997) Methods Enzymol 283, 141-156).

Another modification of the prototypical two-hybrid system includes the reverse two-hybrid method. Reverse two-hybrid methods screen for agents that alter or completely disrupt the intermolecular association between two interacting test polypeptides. When this technique is employed and compared to a control, an agent's inhibitory action towards a particular intermolecular association can be quantified. See, e.g., U.S. Pat. Nos. 5,525,490, 7,601,533, and 7,033,768. Even though the reverse two-hybrid system has proven useful, several problems still exist. In particular, this system analyzes loss of signal when compared to a control. Therefore, an intermolecular association between the two interacting test polypeptides is required before attempting to disrupt the association with an agent. Depending on the strength of the intermolecular association, agents that may otherwise inhibit the initial formation of this association may be overlooked because they fail to disrupt the intermolecular association after its formation. Consequently, screening sensitivity may be an issue when using reverse two-hybrid systems.

It is an object of the invention to provide an improved hybrid system for detecting protein-protein interactions with improved sensitivity and adjustability.

It is a further object of the invention to provide a modified hybrid system that allows binding of candidate inhibitors to bait and/or prey molecules prior to their interaction.

It is another object of the invention to provide methods of identifying agents that prevent protein-protein interactions.

It is a further object of the invention to provide methods for identifying allelic mutations in proteins that lead to decreased drug efficacy. Upon identifying such mutations, it is a further object of the invention to identify new agents that treat and/or inhibit the effects of these newly identified mutations.

### SUMMARY OF THE INVENTION

Controlled release hybrid systems for use in detecting and adjusting protein-protein or protein-DNA interactions are described. Unlike traditional yeast hybrid systems where hybrid pairs begin to interact as soon as they are expressed within the cell, the disclosed systems have the advantage of increased adjustability and sensitivity by controlling the release of one or more of the hybrid pairs in the system.

The controlled release hybrid systems involve a least a host cell expressing and/or containing (1) a detectable reporter gene operably linked to an expression control sequenced, (2) a first hybrid protein containing at least a DNA-binding domain (DBD) that binds the expression control sequence, (3) a second hybrid protein containing at least an activation domain (AD) of a transcriptional activator, (4) a ligand binding domain (LBD) contained in at least one of the hybrid proteins, and (5) a regulatory element that upon binding to the LBD prevents interaction between the first hybrid protein (containing the DBD) and the second hybrid protein (containing the AD).

The first and second hybrid proteins preferably contain heterologous molecules, wherein interaction between the molecule on the first hybrid protein and the molecule on the second hybrid protein results in transcriptional activation of the detectable reporter gene. These molecules are therefore referred to herein as “interacting molecules” “interacting domains” or “interacting pairs.” In preferred embodiments, these molecules are protein sequences. In some embodiments, both of the interacting domains are proteins known to interact. In other embodiments, one of the interacting domains is a known protein and the second interacting domain is from a peptide or chemical library. In these embodiments, the hybrid system can be used to identify peptides or chemicals that bind the first interacting pair. Generally, the known protein is fused to the DBD and the candidate/libary peptide/chemical is fused to the AD.

In preferred embodiments, one of the proteins is a therapeutic target, such as HIV integrase. The second protein can be either a protein that is known to interact with the first protein, e.g., an HIV integrase inhibitor, or a protein from a library of proteins being tested for interaction with the first protein. In other embodiments, both of the interacting pairs are known to interact, e.g., HIV integrase and an inhibitory peptide. In these embodiments, the hybrid system is preferably used to identify agents that inhibit this known interaction.

The interacting pair fused to the DBD is generally referred to herein as “bait” and the interacting pair fused to the AD is generally referred to herein as “prey.” Usually, the bait is a known protein and the prey is a candidate/libary peptide/chemical. However, the terms “bait” and “prey” are used also herein generally to describe interacting pairs in the first and second hybrid proteins, respectively.

The hybrid proteins are therefore at least bipartite in that they contain both (1) a DBD or AD and (2) an interacting domain. Moreover, at least one of the hybrid proteins is tripartite in that it further contains a LBD. In preferred embodiments, only the first hybrid protein contains a LBD, which results in sequestration of the first hybrid protein in the cytoplasm. In other embodiments, only the second hybrid protein contains a LBD. In still other embodiments, both the first and the second hybrid protein contain a LBD.

With the controlled release hybrid systems, successful interaction between the interacting domain attached to the DBD (i.e., bait) and the interacting domain attached to the AD (i.e., prey) results in transactivation of the reporter gene. However, binding of the regulatory element to the LBD prevents this interaction between interacting domains. The controlled release hybrid systems can further involve a ligand capable of displacing the regulatory element from the LBD. This displacement allows the first hybrid protein (containing the DBD) and the second hybrid protein (containing the AD) to interact. Therefore, with this system, ligand availability represents a means for controlling interaction of the interacting domains. This system therefore involves a means for adjusting the timing and/or concentrations of bait-prey interaction.

The LBD is preferably the LBD of a nuclear receptor, such as a type I nuclear receptor. Type I nuclear receptors include androgen receptors, estrogen receptors, glucocorticoid receptors, and progesterone receptors. In these embodiments, the ligand is preferably a steroid that naturally binds the nuclear receptor. Exemplary steroids include gonane derivatives, progestins, androgens, corticosteroids, and anabolic steroid. In preferred embodiments, the ligand is cortisol, hydrocortisone, estrogen, estradiol, estrone, progesterone, testosterone, or a derivative or combination thereof. Therefore, in preferred embodiments, the LBD is an estrogen receptor LBD (“ER^{LBD}”), an androgen receptor LBD (“AR^{LBD}”), a progesterone receptor LBD (“PR^{LBD}”), a testosterone receptor LBD (“TR^{LBD}”), or a glucocorticoid receptor LBD (“GR^{LBD}”).

The regulatory element for these embodiments is preferably a chaperone protein, such as a heat shock protein, that is displaced upon steroid binding. In preferred embodiments, the regulatory element is heat shock protein 90 (Hsp90).

The reporter gene can be any nucleic acid encoding a protein whose expression may be directly or indirectly assayed. In preferred embodiments, the protein is a fluorescent protein (e.g., green fluorescent protein), a luminescent protein (e.g., luciferase), or an enzyme that catalyzes hydrolysis of a substrate into a detectable product (e.g., β-galactosidase, alkaline phosphatases, β-glucuronidase). In some embodiments, the protein confers resistance to a toxin (e.g., antibiotic). In other embodiments, the gene (e.g., URA3, LYS2, ADE2, ADE3, HIS3, TRP1) encodes a protein needed for biosynthesis of a necessary nutrient absent in the growth medium.

In preferred embodiments, the detectable reporter gene encodes β-galactosidase. In these embodiments, the first hybrid protein preferably contains the DNA binding domain of the transcription factor GAL4 (i.e., GAL4^{BD}) and the second hybrid protein preferably contains the transcriptional activation domain of the transcription factor GAL4 (i.e., GAL4^{AD}). In preferred embodiments, the first hybrid protein contains the ligand binding domain of a nuclear receptor, such as the progesterone receptor (PR^{LBD}).

The host cell of the controlled release hybrid system is preferably a cultured eukaryotic cell. In preferred embodiments, the host cell is Saccharomyces cerevisae.

The controlled release hybrid systems can further involve an agent, such as a candidate therapeutic agent. If the agent inhibits binding of the bait and prey in the first and second hybrid proteins, activation of reporter gene expression will be prevented or reduced when the ligand is added to the system. In these embodiments, the system can be used to assay the ability of a candidate agent to prevent the binding of interacting domains as well as assay for the ability to inhibit binding that has already occurred.

In preferred embodiments, the host cell contains a first construct containing a recombinant nucleic acid sequence encoding the first hybrid protein and a second construct containing a recombinant nucleic acid sequence encoding the second hybrid protein. The recombinant nucleic acid sequences are each preferably operably linked to an expression control sequence sufficient to activate expression of the hybrid proteins in the host cell. The first and second constructs can be present in the same polynucleotide, e.g., plasmid, but are preferably present in distinct polynucleotides.

Kits containing polynucleotides (e.g., plasmids) encoding one or more of a first hybrid protein, a second hybrid protein, and a regulatory element as described above are provided. The kits can further contain one or more maps (e.g., restriction map) of the one or more constructs with instructions for inserting nucleic acids encoding proteins of interest into the polynucleotides in frame, e.g., using a restriction endonuclease.

The individual components of the kits can be packaged in a variety of containers, e.g., vials, tubes, microtiter well plates, bottles, and the like. Other reagents can be included in separate containers and provided with the kit; e.g., positive control samples, negative control samples, buffers, cell culture media, etc. Preferably, the kits will also include instructions for use.

A method for identifying agents that reduce or prevent intermolecular binding between interacting proteins is also provided. The first step of the method involves administering a candidate agent to a host cell expressing and/or containing the first and second hybrid proteins described above and expressing or containing a regulatory element that prevents interaction of the first and second hybrid proteins, wherein the first and second hybrid proteins contain interacting domains. The second step of the method involves administering to the cell a ligand that is capable of displacing the regulatory element in the hybrid system. In this step, of the method, displacing the regulatory element allows the first hybrid protein to interact with the second hybrid protein. The third step of the method involves assaying the host cell for expression of the reporter gene and comparing the expression to a control. In this step, reduced reporter expression in the host cell compared to a control is an indication that the candidate agent reduces intermolecular binding between interacting domains

In some embodiments, the controlled release hybrid system is used to screen for interactions between candidate molecules, such as proteins. In preferred embodiments, the controlled release hybrid system is used to screen for chemical inhibitors of the interacting candidate molecules. In particular, these chemical inhibitors can disrupt the molecular interaction of an interacting pair (e.g., protein-protein binding). In some embodiments, the controlled release hybrid system is used to screen for mutant proteins whose interaction with another protein is no longer inhibited with existing chemical inhibitors. These mutant proteins can be further assayed for new chemical inhibitors that successfully inhibit this interaction.

Use of the controlled release systems described above for personalized medication applications is also disclosed.

### BRIEF DESCRIPTION OF THE DRAWINGS

FIG. 1 displays a representative schematic of the controlled release two hybrid system.

FIG. 2 shows a representative tripartite construct having a DNA binding domain (e.g., GALDBD), a ligand binding domain (e.g., progesterone ligand binding domain—(PR)LBD), and a prey domain (e.g., InF5 peptide) under the control of an ADH promoter.

FIG. 3 shows a progesterone dose response curve for the progesterone dependent HIV integrase peptide two hybrid interaction in yeast.

FIG. 4 shows a progesterone dose response curve to a control yeast strain.

### DETAILED DESCRIPTION OF THE INVENTION

### I. Definitions

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within the ranges as if each numerical value and sub-range is explicitly recited. As an illustration, a numerical range of “about 1 to 5” should be interpreted to include not only the explicitly recited values of about 1 to about 5, but also include individual values such as 2, 3, and 4 and sub-ranges such as from 1-3, from 2-4, and from 3-5, etc. as well as 1, 2, 3, 4, and 5, individually. The same principle applies to ranges reciting only one numerical value as a minimum or maximum. Furthermore, such an interpretation should apply regardless of the breadth of the range or the characteristics being described.

It must be noted that as used herein and in the appended claims, the singular forms “a,” “an,” and “the” include plural referents unless the context clearly dictates otherwise. Thus, for example, reference to “a ligand” includes a plurality of such ligands, reference to “the ligand” is a reference to one or more ligands and equivalents thereof known to those skilled in the art, and so forth.

Ranges can be expressed herein from “about” one particular value, and/or to “about” another particular value. When such a range is expressed, another embodiment includes from the one particular value and/or to the other particular value. Similarly, when values are expressed as approximations, by used of the antecedent “about,” it will be understood that the particular value forms another embodiment. It will further understood that the endpoints of each of the ranges are significant both in relation to the endpoint, and independently of the other endpoint. It is also understood that there are a number of values disclosed herein, and that each value is also herein disclosed as “about” that particular value in addition to the value itself. For example, if the value “10” is disclosed, the “about” 10 is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application, data are provided in a number of different formats, and that these data represent endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

The term “hybrid system” as used herein refers to a molecular biology technique used to detect interactions between interacting pairs (e.g., bait and prey proteins) that involves the use of one or more hybrid proteins that activate expression of a detectable reporter gene when such interaction occurs. Hybrid systems include one-hybrid systems, a two-hybrid systems, and a three-hybrid systems.

The term “controlled release” refers to the ability to control the interaction of interacting domains in a hybrid system in a concentration dependent manner, a time dependent manner, or a combination thereof.

The term “reporter gene” refers to a gene that encodes a protein (e.g., enzyme) whose expression may be assayed and is operably linked to an expression control sequence that is transactivated by a transcriptional activator (e.g., transcription factor) that can be functionally separated into a DNA binding domain and an activation domain.

The term “host cell” refers to a cell suitable for detectable expression of the reporter gene in culture. Suitable host cells include prokaryotic cells (e.g., a bacterial cell such as E. coli), lower eukaryotic cells, and higher eukaryotic cells. Examples of lower eukaryotic cells include, but are not limited to, yeast such as Saccharomyces cerevisiae, or genetically modified strains thereof. Examples of higher eukaryotic cells include, but are not limited to, primary or transformed animal cell lines (e.g., mammalian cells, CHO cells, MEF cells, chicken DT40 cells, normal human fibroblasts, etc.) that are capable of being cultured.

The term “vector” or “construct” refers to a nucleic acid sequence capable of transporting into a cell another nucleic acid to which the vector sequence has been linked. The construct can include, but is not limited to, a plasmid that encodes for and expresses at least one heterologous nucleic acid sequence.

The term “expression control sequence” refers to a cis-regulatory element that regulates transcription of a gene. Promoters are examples of expression control sequences that are generally located 5′ to the transcription start site. Promoters contain specific DNA sequences and response elements which provide a secure initial binding site for RNA polymerase and for transcription factors that recruit RNA polymerase.

The term “operably linked” refers to the physical and functional relationship of a nucleic acid with another nucleic acid sequence. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “transformation” and “transfection” refer to the introduction of a nucleic acid, e.g., a plasmid, into a recipient cell. For genetic expression, the nucleic acid is preferably integrated into the chromosomal DNA of the cell.

A “chimeric molecule” is a single molecule created by joining two or more molecules that exist separately in their native state.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. Fusion proteins can be prepared using conventional techniques in molecular biology to join the two genes in frame into a single nucleic acid, and then expressing the nucleic acid in an appropriate host cell under conditions in which the fusion protein is produced.

The term “hybrid protein” refers to a fusion protein or a chimeric molecule containing a DNA binding domain or transcriptional activation domain from a transcriptional regulatory protein (e.g., transcription factor) linked to a heterologous molecule (e.g., protein) to be assayed for interaction.

The term “protein domain” refers to a portion of a protein, portions of a protein, or an entire protein showing structural integrity. For example, HIV integrase may be divided into at least three domains, which include an N-terminus portion, a central core, and a C-terminus. The GAL4 transcription factor may be divided into at least two domains, the DNA binding domain (“BD”) and the transcription activation domain (“AD”).

The terms “peptide,” “polypeptide,” and “protein” may be used interchangeably to refer to a natural or synthetic molecule having two or more amino acids linked by the carboxyl group of one amino acid to the alpha amino group of another.

The term “mutant” or “variant” refers to a nucleic acid sequence or amino acid sequence having changes (i.e., substitutions, additions, deletions, etc.) in the nucleic acid sequence or amino sequence respectively when compared to a wild type sequence. For example, an amino acid or peptide sequence mutant or variant can have conservative amino acid substitutions, non-conservative amino acid substitutions (i.e., a degenerate variant), substitutions within the wobble position of each codon encoding an amino acid, amino acids added to the N-terminus, amino acids added to the C-terminus, or a combination thereof of a peptide sequence, deletion of amino acid(s), or a peptide having 30%, 40%, 50%, 60%, 70%, 80%, 90%, 95%, or 99% sequence identity to a wild type amino acid sequence.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods. For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

The term “regulatory element” as it applies to a disclosed hybrid protein refers to a molecule (e.g., a synthetic or naturally occurring molecule) capable of binding a ligand binding domain (LBD) in a hybrid protein and preventing the hybrid protein from interacting with a second hybrid protein.

The term “ligand” as it applies to the disclosed hybrid proteins refers to a molecule (e.g., synthetic molecule or naturally occurring molecule) capable of displacing a regulatory element from a LBD on a hybrid protein thereby allowing the hybrid protein to interacting with a second hybrid protein.

The term “ligand binding domain” or “LBD” as it applies to a disclosed hybrid protein refers to any protein sequence that is a binding site for a regulatory element and a ligand wherein ligand binding displaces the regulatory element.

The term “displace” refers to the ability of a ligand to bind a LBD and thereby reduce the interaction of a regulatory element with the LBD in a concentration dependent manner.

The term “steroid” refers to synthetic hormone compounds, naturally occurring hormone compounds, or a combination thereof that bind ligand binding domains of nuclear receptors. Steroids can include, but are not limited to, androgens (e.g., testosterone, estrogen, etc.), progestins, corticosteroids, anabolic steroids, ecdysteroids, plant sterols, ergosterols, or any combination thereof.

The term “interaction” or “molecular interaction” refers to stable or transient non-covalent bonding of two molecules. The non-covalent bonds can be ionic bonds, hydrogen bonds, electrostatic interactions, van der Waals forces, metal ion binding, or combinations thereof. In certain embodiments, the interacting pair can indirectly bind one another (e.g., two proteins independently bind a co-factor, metal or nucleic acid thus creating an interaction). The term “interaction” does not denote a particular affinity of the interaction. However, in preferred embodiments, an interaction that is detectable by the disclosed systems and methods involves a binding affinity constant (Ka) greater than about 10⁴M⁻¹ (e.g., 10⁴M⁻¹, 10⁵M⁻¹, 10⁶M⁻¹, or more) between the interacting pairs. For example, in some embodiments, the peptide inhibitor of HIV integrase (SEQ ID NO:7) binds HIV integrase with a Kd of about 5×10⁻⁶ M (i.e., Ka of about 2×10⁵ M⁻¹), which can be detected by the disclosed methods.

The term “inhibiting” or “reducing” an interaction means to lower the average interaction between two molecules by at least 5%, 10%, 20%, 30%, 40%, 50%, 60%, 70%, 80%, 90%, or 99% when compared to a positive control.

The term “preventing” an interaction does not require absolute forestalling but includes reducing the occurrence of the interaction between two molecules before it occurs by at least 5%, 10%, 20%, 30%, 40%, 50%, 60%, 70%, 80%, 90%, or 99% when compared to a positive control. An agent that inhibits interactions will also prevent the interaction by at least the same amount. However, an agent that is unable to substantially inhibit an existing interaction may in some embodiments be able to inhibit occurrence of the interaction.

### II. Controlled Release Hybrid Systems

Controlled release hybrid systems are described for detecting interactions between molecules, such as proteins. For example, the controlled release hybrid systems can be used to detect binding of a molecule, e.g., peptide, to a therapeutic target, such as HIV integrase. Moreover, the controlled release hybrid systems can be used to detect the ability of an agent, e.g, a chemical compound, to inhibit this interaction.

In certain embodiments, the controlled release hybrid system is a one-hybrid system, a two-hybrid system, or a three-hybrid system. In certain embodiments, the controlled release hybrid system is a reverse hybrid system. Furthermore, in certain embodiments, the controlled release hybrid system includes a host cell expressing and/or containing at least a (1) detectable reporter-gene, (2) a first hybrid protein expressed from at least a first bipartite construct, and a second hybrid protein expressed from at least a second bipartite construct, wherein at least either the first hybrid protein or the second hybrid protein is expressed from a tripartite construct, and (3) a regulatory element that binds to the hybrid protein expressed from the tripartite construct and prevents an interaction between the first hybrid protein and the second hybrid protein thus preventing the activation of the detectable reporter gene.

When assaying for potential molecular interactions between the first and second hybrid protein (i.e., interacting domains or potential interacting domains), it is desirable to allow the first and second hybrid protein to interact in a controlled and continuously adjustable manner. This may be accomplished by altering the availability of the first hybrid protein, second hybrid protein, or a combination thereof. In preferred embodiments, the availability of the first hybrid protein, the second hybrid protein, or a combination thereof can be altered by administration of a ligand. In these embodiments, the ligand allows the first and second hybrid protein to interact in a controlled manner when administered to the host cell.

In preferred embodiments, the ligand displaces a regulatory element from the hybrid protein that was expressed from the tripartite construct. Upon displacing the regulatory element, the ligand binds to the hybrid protein derived from the tripartite construct and allows the first hybrid protein and second hybrid protein to interact. When the first hybrid protein and second hybrid protein interact, activation of the detectable reporter gene occurs. By controlling the availability of the first hybrid protein, second hybrid protein, or a combination thereof through the administration of a ligand, the interaction between the first and second hybrid protein is also controlled. Likewise, by controlling the interaction between the first and second hybrid protein, output of the system via reporter-gene expression can also be controlled.

A. Reporter-Genes

The controlled release hybrid systems include at least one detectable reporter-gene. Reporter-genes can include, but are not limited to beta-galactosidase (LacZ), Beta-glucuronidase (GUS), alkaline phosphatase, amino acid biosynthetic genes (e.g., yeast LEU2, HIS3, or LYS2 genes), nucleic acid biosynthetic genes (e.g., URA3 or ADE2 genes), the chloramphenicol acetyltransferase (CAT) gene, the green fluorescent protein (GFP) gene, the yellow fluorescent protein (YFP), the red fluorescent protein, orange fluorescent protein, luciferase, or any combination thereof. In certain embodiments, the reporter-gene may be provided to the host cell either before or after the constructs that express the hybrid proteins have been provided to the host cell. In preferred embodiments, the reporter-gene is provided to the host cell before the constructs that express the hybrid proteins. For example, the host cell can be either transformed or transfected with the reporter-gene using methods various methods known in the art (e.g., electroporation, chemical based methods, microinjection, etc.).

Suitable detectable reporter genes encode a protein whose expression can be directly or indirectly assayed. In preferred embodiments, the protein is a fluorescent protein, a luminescent protein, an enzyme that catalyzes hydrolysis of a substrate into a detectable product, confers resistance to a toxin, or is needed for biosynthesis of a necessary nutrient absent in the growth medium.

In preferred embodiments, the reporter-gene is operably linked to at least one expression control sequence that is transactivated by a transcription factor that functions when split into a DNA binding domain (DBD) on one peptide and an activation domain (AD) on a separate peptide. This expression control sequence is preferably located such that if a first hybrid protein having a DBD of the transcription factor binds to a DNA binding site and interacts with a second hybrid protein having an activation domain, the activation domain of the second hybrid protein will be able to activate transcription of the reporter-gene.

B. Constructs

The controlled release hybrid systems utilize numerous bipartite and tripartite constructs. In certain embodiments, the bipartite or tripartite constructs include at least a (1) DNA binding domain (DBD) or an activation domain (AD) [but preferably not both] and (2) a heterologous sequence encoding bait or prey molecules (e.g., proteins). In addition, the tripartite constructs further contain (3) a ligand binding domain (LBD).

In certain embodiments, the DNA binding domain (DBD) includes, but is not limited to, a protein domain capable of binding to an expression control sequence operably linked to the reporter gene. Examples of DBDs include, but are not limited to, LexA and GAL4^{DBD}.

The following is an exemplary nucleic acid sequence encoding GAL4^{DBD}:

(SEQ ID NO: 1)
ATGAAGCTACTGTCTTCTATCGAACAAGCATGCGATATTTGCCGACT
TAAAAAGCTCAAGTGCTCCAAAGAAAAACCGAAGTGCGCCAAGT
GTCTGAAGAACAACTGGGAGTGTCGCTACTCTCCCAAAACCAAAA
GGTCTCCGCTGACTAGGGCACATCTGACAGAAGTGGAATCAAGGC
TAGAAAGACTGGAACAGCTATTTCTACTGATTTTTCCTCGAGAAGA
CCTTGACATGATTTTGAAAATGGATTCTTTACAGGATATAAAAGCAT
TGTTAACAGGATTATTTGTACAAGATAATGTGAATAAAGATGCCGTC
ACAGATAGATTGGCTTCAGTGGAGACTGATATGCCTCTAACATTGA
GACAGCATAGAATAAGTGCGACATCATCATCGGAAGAGAGTAGTA
ACAAAGGTCAAAGACAGTTGACTGTATCGCCGGTATTGCAATACCC
AGCTTTGACT.

The following is an exemplary amino acid sequence for GAL4^{DBD}:

(SEQ ID NO: 2)
MKLLSSIEQACDICRLKKLKCSKEKPKCAKCLKNNWECRYSPKTKRS
PLTRAHLTEVESRLERLEQLFLLIFPREDLDMILKMDSLQDIKALLTGL
FVQDNVNKDAVTDRLASVETDMPLTLRQHRISATSSSEESSNKGQRQ
LTVSPVLQYPALT.

In certain embodiments, the DBD includes, but is not limited to, SEQ ID NO:2, or any variant thereof having at least east 65%, 70%, 75%, 80%, 85%, 90%, 95%, or 99% sequence homology to SEQ ID NO:2 that binds the LacZ expression control sequence.

In certain embodiments, the activation domain (AD) includes, but is not limited to, a protein domain capable inducing transcription of a reporter-gene when brought in close proximity or upon interacting with the DBD. Examples of ADs include, but are not limited to, GAL4^{AD}, VP16, and B42.

The following is an exemplary nucleic acid sequence encoding GAL4^{AD}:

(SEQ ID NO: 3)
ATGGATAAAGCGGAATTAATTCCCGAGCCTCCAAAAAAGAAGAGA
AAGGTCGAATTGGGTACCGCCGCCAATTTTAATCAAAGTGGGAATA
TTGCTGATAGCTCATTGTCCTTCACTTTCACTAACAGTAGCAACGG
TCCGAACCTCATAACAACTCAAACAAATTCTCAAGCGCTTTCACAA
CCAATTGCCTCCTCTAACGTTCATGATAACTTCATGAATAATGAAAT
CACGGCTAGTAAAATTGATGATGGTAATAATTCAAAACCACTGTCA
CCTGGTTGGACGGACCAAACTGCGTATAACGCGTTTGGAATCACTA
CAGGGATGTTTAATACCACTACAATGGATGATGTATATAACTATCTAT
TCGATGATGAAGATACCCCACCAAACCCAAAAAAAGAGATCTTTAA
TACGACTCACTATAGGGCGAGCGCCGCCATGGAGTACCCATACGAC
GTACCAGATTACGCTCATATG.

The following is an exemplary amino acid sequence for GAL4^{AD}:

(SEQ ID NO: 4)
MDKAELIPEPPKKKRKVELGTAANFNQSGNIADSSLSFTFTNSSNGPN
LITTQTNSQALSQPIASSNVHDNFMNNEITASKIDDGNNSKPLSPGWT
DQTAYNAFGITTGMFNTTTMDDVYNYLFDDEDTPPNPKKEIFNTTHY
RASAAMEYPYDVPDYA

In certain embodiments, the AD includes, but is not limited to, SEQ ID NO:4, or any variant thereof having at least east 65%, 70%, 75%, 80%, 85%, 90%, 95%, or 99% sequence homology to SEQ ID NO:4 that transactivates transcription of the reporter gene.

In preferred embodiments, the LBD is a protein domain capable of specifically binding to both (1) regulatory elements that can prevent the interaction between the first and second hybrid proteins and (2) ligands that can displace the regulatory elements. Displacement of the regulatory element and binding of the ligand subsequently allows for the first hybrid protein to interact with the second hybrid protein.

In preferred embodiments, the LBD is from a steroid hormone receptor and the ligand is a steroid hormone or steroid-like compound that binds the steroid hormone receptor. Examples of LBDs include, but are not limited to, LBDs derived from progesterone receptors (PR), estrogen receptors (ER), glucocorticoid receptors (GR), androgen receptors (AR), mineralocorticoid receptors (MR), all-trans retinoic acid receptors (RAR), 9-cis retinoic acid receptors (RXR), thyroid hormone receptors, ecdysone receptors (EcR), orphan receptors, or any combination thereof.

The following is an exemplary nucleic acid sequence encoding progesterone receptor LBD (PR^{LBD}):

(SEQ ID NO: 5)
ATGATACAGTTGATTCCACCACTGATCAACCTGTTAATGAGCATTGA
ACCAGATGTGATCTATGCAGGACATGACAACACAAAACCTGACAC
CTCCAGTTCTTTGCTGACAAGTCTTAATCAACTAGGCGAGAGGCAA
CTTCTTTCAGTAGTCAAGTGGTCTAAATCATTGCCAGGTTTTCGAA
ACTTACATATTGATGACCAGATAACTCTCATTCAGTATTCTTGGATG
AGCTTAATGGTGTTTGGTCTAGGATGGAGATCCTACAAACATGTCA
GTGGGCAGATGCTGTATTTTGCACCTGATCTAATACTAAATGAACA
GCGGATGAAAGAATCATCATTCTATTCATTATGCCTTACCATGTGGC
AGATCCCACAGGAGTTTGTCAAGCTTCAAGTTAGCCAAGAAGAGT
TCCTCTGTATGAAAGTATTGTTACTTCTTAATACAATTCCTTTGGAA
GGGCTACGAAGTCAAACCCAGTTTGAGGAGATGAGGTCAAGCTAC
ATTAGAGAGCTCATCAAGGCAATTGGTTTGAGGCAAAAAGGAGTT
GTGTCGAGCTCACAGCGTTTCTATCAACTTACAAAACTTCTTGATA
ACTTGCATGATCTTGTCAAACAACTTCATCTGTACTGCTTGAATACA
TTTATCCAGTCCCGGGCACTGAGTGTTGAATTTCCAGAAATGATGT
CTGAAGTTATTGCTGCACAATTACCCAAGATATTGGCAGGGATGGT
GAAACCCCTTCTCTTTCATAAAGCC.

The following is an exemplary amino acid sequence for PR^{LBD}:

(SEQ ID NO: 6)
MIQLIPPLINLLMSIEPDVIYAGHDNTKPDTSSSLLTSLNQLGERQLLSV
VKWSKSLPGFRNLHIDDQITLIQYSWMSLMVFGLGWRSYKHVSGQM
LYFAPDLILNEQRMKESSFYSLCLTMWQIPQEFVKLQVSQEEFLCMK
VLLLLNTIPLEGLRSQTQFEEMRSSYIRELIKAIGLRQKGVVSSSQRFY
QLTKLLDNLHDLVKQLHLYCLNTFIQSRALSVEFPEMMSEVIAAQLPK
ILAGMVKPLLFHKA.

The following is an exemplary nucleic acid sequence encoding androgen receptor LBD (AR^{LBD}):

(SEQ ID NO: 13)
ATGTGTCAGCCCATCTTTCTGAATGTCCTGGAAGCCATTGAG
CCAGGTGTAGTGTGTGCTGGACACGACAACAACCAGCCCGACTCC
TTTGCAGCCTTGCTCTCTAGCCTCAATGAACTGGGAGAGAGACAG
CTTGTACACGTGGTCAAGTGGGCCAAGGCCTTGCCTGGCTTCCGC
AACTTACACGTGGACGACCAGATGGCTGTCATTCAGTACTCCTGGA
TGGGGCTCATGGTGTTTGCCATGGGCTGGCGATCCTTCACCAATGT
CAACTCCAGGATGCTCTACTTCGCCCCTGATCTGGTTTTCAATGAGT
ACCGCATGCACAAGTCCCGGATGTACAGCCAGTGTGTCCGAATGA
GGCACCTCTCTCAAGAGTTTGGATGGCTCCAAATCACCCCCCAGG
AATTCCTGTGCATGAAAGCACTGCTACTCTTCAGCATTATTCCAGTG
GATGGGCTGAAAAATCAAAAATTCTTTGATGAACTTCGAATGAACT
ACATCAAGGAACTCGATCGTATCATTGCATGCAAAAGAAAAAATCC
CACATCCTGCTCAAGACGCTTCTACCAGCTCACCAAGCTCCTGGAC
TCCGTGCAGCCTATTGCGAGAGAGCTGCATCAGTTCACTTTTGACC
TGCTAATCAAGTCACACATGGTGAGCGTGGACTTTCCGGAAATGAT
GGCAGAGATCATCTCTGTGCAAGTGCCCAAGATCCTTTCTGGGAA
AGTCAAGCCCATCTATTTCCACACCGCG.

The following is an exemplary amino acid sequence for AR^{LBD}:

(SEQ ID NO: 14)
CQPIFLNVLEAIEPGVVCAGHDNNQPDSFAALLSSLNELGERQ
LVHVVKWAKALPGFRNLHVDDQMAVIQYSWMGLMVFAMGWRSFT
NVNSRMLYFAPDLVFNEYRMHKSRMYSQCVRMRHLSQEFGWLQITP
QEFLCMKALLLFSIIPVDGLKNQKFFDELRMNYIKELDRIIACKRKNP
TSCSRRFYQLTKLLDSVQPIARELHQFTFDLLIKSHMVSVDFPEMMAE
IISVQVPKILSGKVKPIYFHTA.

The following is an exemplary nucleic acid sequence encoding estrogen receptor LBD (ER^{LBD}):

(SEQ ID NO: 15)
ATGCCTATCGATATCAGTCGAGCTTCTGCTGGAGACATGAGAGCTG
CCAACCTTTGGCCAAGCCCGCTCATGATCAAACGCTCTAAGAAGA
ACAGCCTGGCCTTGTCCCTGACGGCCGACCAGATGGTCAGTGCCT
TGTTGGATGCTGAGCCCCCCATACTCTATTCCGAGTATGATCCTACC
AGACCCTTCAGTGAAGCTTCGATGATGGGCTTACTGACCAACCTG
GCAGACAGGGAGCTGGTTCACATGATCAACTGGGCGAAGAGGGT
GCCAGGCTTTGTGGATTTGACCCTCCATGATCAGGTCCACCTTCTA
GAATGTGCCTGGCTAGAGATCCTGATGATTGGTCTCGTCTGGCGCT
CCATGGAGCACCCAGTGAAGCTACTGTTTGCTCCTAACTTGCTCTT
GGACAGGAACCAGGGAAAATGTGTAGAGGGCATGGTGGAGATCTT
CGACATGCTGCTGGCTACATCATCTCGGTTCCGCATGATGAATCTGC
AGGGAGAGGAGTTTGTGTGCCTCAAATCTATTATTTTGCTTAATTCT
GGAGTGTACACATTTCTGTCCAGCACCCTGAAGTCTCTGGAAGAG
AAGGACCATATCCACCGAGTCCTGGACAAGATCACAGACACTTTG
ATCCACCTGATGGCCAAGGCAGGCCTGACCCTGCAGCAGCAGCAC
CAGCGGCTGGCCCAGCTCCTCCTCATCCTCTCCCACATCAGGCACA
TGAGTAACAAAGGCATGGAGCATCTGTACAGCATGAAGTGCAAGA
ACGTGGTGCCCCTCTATGACCTGCTGCTGGAGATGCTGGACGCCCA
CCGCCTACATGCGCCCACTAGCCGTGGAGGGGCATCCGTGGAGGA
GACGGACCAAAGCCACTTGGCCACTGCGGGCTCTACTTCATCGAT
GATCACGGCG.

The following is an exemplary amino acid sequence for ER^{LBD}:

(SEQ ID NO: 16)
PIDISRASAGDMRAANLWPSPLMIKRSKKNSLALSLTADQMVSALLD
AEPPILYSEYDPTRPFSEASMMGLLTNLADRELVHMINWAKRVPGFV
DLTLHDQVHLLECAWLEILMIGLVWRSMEHPVKLLFAPNLLLDRNQG
KCVEGMVEIFDMLLATSSRFRMMNLQGEEFVCLKSIILLNSGVYTFLS
STLKSLEEKDHIHRVLDKITDTLIHLMAKAGLTLQQQHQRLAQLLLIL
SHIRHMSNKGMEHLYSMKCKNVVPLYDLLLEMLDAHRLHAPTSRGG
ASVEETDQSHLATAGSTSSMITA.

Examples of ligand binding domains include variants, or fragments thereof, having at least east 65%, 70%, 75%, 80%, 85%, 90%, 95%, or 99% sequence homology to SEQ ID NO:6, SEQ ID NO:16, or SEQ ID NO:14 that maintain affinity for there respective hormone receptor. In certain embodiments, the LBD includes only mammalian steroid receptors or portions of mammalian steroid receptors (e.g., mammalian ligand binding domains). In other embodiments, the LBD includes only human steroid receptors or portions thereof. In certain embodiments, the LBD does not include insect hormone receptors. In certain embodiments, the LBD is SEQ ID NO:6, or any fragment or variant thereof having at least east 65%, 70%, 75%, 80%, 85%, 90%, 95%, or 99% sequence homology to SEQ ID NO:6 that binds progesterone with high affinity.

Domains for use in the disclosed contructs can be modified to include additional sequences for use in directed cloning of the domains into contructs. For example, the nucleic acid encoding the LBD, AD, DBD, or interacting peptides can further contain restriction endonuclease sites that allow ligation of the nucleic acid into expression contructs in frame and operably linked to an expression control sequence.

In certain embodiments, the controlled release hybrid systems can be used to screen for molecules, e.g., proteins, that interact with one another. In certain embodiments, these interacting molecules can be incorporated into the constructs described herein and used as either prey (P) domains, bait (B) domains, or any combination thereof. Prey/Bait (P/B) refers to a portion of a hybrid protein that can form a specific binding interaction with a portion of a second hybrid protein under suitable binding conditions. Generally, a portion of the first hybrid protein preferentially binds to a portion of the second hybrid protein forming a heterodimer or higher order heteromultimer including the first and second hybrid proteins. The binding portions of each hybrid protein are termed “interacting pair,” “interacting molecules,” “interacting domains,” “interacting protein.” In addition, the interacting pair are referred to as “bait” and “prey” molecules to reflect the ability to identify a binding pair from a peptide or chemical library. These terms may be used interchangeably throughout the specification and claims. In certain embodiments, examples of known interacting pairs include, but are not limited to, HIVGag-Gag, HPV16-peptide binders, and RB-E7.

Numerous bipartite and tripartite constructs may be created by mixing and matching various sequences encoding for a DBD, LBD, AD, peptides, polypeptides or portions thereof, protein or portions thereof, or any combination thereof. The bipartite and tripartite constructs, include but are not limited to the examples disclosed below:

(1) Bipartite Constructs

As described herein, the controlled release hybrid system can include a bipartite construct. In certain embodiments, the bipartite construct can include a DNA Binding Domain (DBD) or activation domain (AD); and an interacting domain (e.g., bait or prey peptide).

In certain embodiments, the bipartite construct includes, but is not limited to, a sequence encoding for GAL4^{DBD}-bait or GAL4^{AD}-prey. In one embodiment, the bait/prey pair is a peptide inhibitor that binds HIV integrase and HIV integrase, or a fragment thereof that binds the peptide inhibitor, e.g., amino acids 1-210 of HIV integrase. For example, in this embodiment, the bipartite construct can be GAL4^{DBD}-HIV integrase (or a fragment thereof) or GAL4^{DBD}-HIV integrase inhibiting peptide.

In some embodiments, the HIV integrase inhibiting peptide can include, but is not limited to SEQ ID NO:7 (LYETILILLFLDVDT), an amino acid sequence encoded by the nucleic acid sequence SEQ ID NO:8 (TTGTACGAGACTATCTTGATTTTGCTGTTTCTTGACGTGGATACG), or any variant or fragment thereof that binds HIV integrase. For example, variants include amino acid sequences have at least 65%, 70%, 75%, 80%, 85%, 90%, 95%, or 99% sequence homology to SEQ ID NO:7 or to an amino acid encoded by SEQ ID NO:8.

The following is an exemplary nucleic acid sequence encoding amino acids 1-210 of HIV integrase:

(SEQ ID NO: 9)
ACTTTTTAGATGGAATAGATAAGGCCCAAGATGAACATGAGAAATA
TCACAGTAATTGGAGAGCAATGGCTAGTGATTTTAACCTGCCACCT
GTAGTAGCAAAAGAAATAGTAGCCAGCTGTGATAAATGTCAGCTAA
AAGGAGAAGCCATGCATGGACAAGTAGACTGTAGTCCAGGAATAT
GGCAACTAGATTGTACACATTTAGAAGGAAAAGTTATCCTGGTAGC
AGTTCATGTAGCCAGTGGATATATAGAAGCAGAAGTTATTCCAGCA
GAAACAGGGCAGGAAACAGCATATTTTCTTTTAAAATTAGCAGGA
AGATGGCCAGTAAAAACAATACATACAGACAATGGCAGCAATTTC
ACCAGTGCTACGGTTAAGGCCGCCTGTTGGTGGGCGGGAATCAAG
CAGGAATTTGGAATTCCCTACAATCCCCAAAGTCAAGGAGTAGTAG
AATCTATGAATAAAGAATTAAAGAAAATTATAGGACAGGTAAGAGA
TCAGGCTGAACATCTTAAGACAGCAGTACAAATGGCAGTATTCATC
CACAATTTTAAAAGAAAAGGGGGGATTGGGGGGTACAGTGCAGGG
GAAAGAATAGTAGACATAATAGCAACAGACATACAAACT.

The following is an exemplary amino acid sequence for amino acids 1-210 of HIV integrase:

(SEQ ID NO: 10)
FLDGIDKAQDEHEKYHSNWRAMASDFNLPPVVAKEIVASCDKCQLK
GEAMHGQVDCSPGIWQLDCTHLEGKVILVAVHVASGYIEAEVIPAET
GQETAYFLLKLAGRWPVKTIHTDNGSNFTSATVKAACWWAGIKQEF
GIPYNPQSQGVVESMNKELKKIIGQVRDQAEHLKTAVQMAVFIHNFK
RKGGIGGYSAGERIVDIIATDIQT.

The following is an exemplary nucleic acid sequence encoding full length HIV integrase:

(SEQ ID NO: 11)
TTTTTAGATGGAATAGATAAGGCCCAAGAAGAACATGAGAAATATC
ACAGTAATTGGAGAGCAATGGCTAGTGATTTTAACCTACCACCTGT
AGTAGCAAAAGAAATAGTAGCCAGCTGTGATAAATGTCAGCTAAA
AGGGGAAGCCATGCATGGACAAGTAGACTGTAGCCCAGGAATATG
GCAGCTAGATTGTACACATTTAGAAGGAAAAGTTATCTTGGTAGCA
GTTCATGTAGCCAGTGGATATATAGAAGCAGAAGTAATTCCAGCAG
AGACAGGGCAAGAAACAGCATACTTCCTCTTAAAATTAGCAGGAA
GATGGCCAGTAAAAACAGTACATACAGACAATGGCAGCAATTTCA
CCAGTACTACAGTTAAGGCCGCCTGTTGGTGGGCGGGGATCAAGC
AGGAATTTGGCATTCCCTACAATCCCCAAAGTCAAGGAGTAATAGA
ATCTATGAATAAAGAATTAAAGAAAATTATAGGACAGGTAAGAGAT
CAGGCTGAACATCTTAAGACAGCAGTACAAATGGCAGTATTCATCC
ACAATTTTAAAAGAAAAGGGGGGATTGGGGGGTACAGTGCAGGG
GAAAGAATAGTAGACATAATAGCAACAGACATACAAACTAAAGAA
TTACAAAAACAAATTACAAAAATTCAAAATTTTCGGGTTTATTACA
GGGACAGCAGAGATCCAGTTTGGAAAGGACCAGCAAAGCTCCTCT
GGAAAGGTGAAGGGGCAGTAGTAATACAAGATAATAGTGACATAA
AAGTAGTGCCAAGAAGAAAAGCAAAGATCATCAGGGATTATGGAA
AACAGATGGCAGGTGATGATTGTGTGGCAAGTAGACAGGATGAGG
TAAGGATCCATCGAGCTCGAGCTGCAGATGAATCG

The following is an exemplary amino acid sequence for full length HIV integrase:

(SEQ ID NO: 12)
FLDGIDKAQEEHEKYHSNWRAMASDFNLPPVVAKEIVASCDK
CQLKGEAMHGQVDCSPGIWQLDCTHLEGKVILVAVHVASGYIEAEVI
PAETGQETAYFLLKLAGRWPVKTVHTDNGSNFTSTTVKAACWWAGI
KQEFGIPYNPQSQGVIESMNKELKKIIGQVRDQAEHLKTAVQMAVFIH
NFKRKGGIGGYSAGERIVDIIATDIQTKELQKQITKIQNFRVYYRDSRD
PVWKGPAKLLWKGEGAVVIQDNSDIKVVPRRKAKIIRDYGKQMAGD
DCVASRQDEVRIHRARAADES.

In some embodiments, the portion of HIV integrase includes at least SEQ ID NO:10, or any variant or fragment thereof having at least east 65%, 70%, 75%, 80%, 85%, 90%, 95%, or 99% sequence homology to SEQ ID NO:10 or an amino acid encoded by SEQ ID NO:9 that binds the peptide inhibitor that binds HIV integrase. In some embodiments, the portion of HIV integrase comprises SEQ ID NO:12, or a variant thereof having at least east 65%, 70%, 75%, 80%, 85%, 90%, 95%, or 99% sequence homology to SEQ ID NO:12 or an amino acid encoded by SEQ ID NO:11.

In certain embodiments, the bipartite construct includes, but is not limited to a sequence encoding for Gal4-AD-peptide (e.g., Gal4-AD-HIV integrase inhibiting peptide). In this embodiment, the HIV integrase peptide can include, but is not limited to, SEQ ID NO:9, SEQ ID NO:10, SEQ ID NO:11, SEQ ID NO:12, or any variant thereof having at least east 65%, 70%, 75%, 80%, 85%, 90%, 95%, or 99% sequence homology to SEQ ID NO:9, SEQ ID NO:10, SEQ ID NO:11, or SEQ ID NO:12. In certain embodiments, the bipartite construct includes a sequence encoding for Gal4-AD-a portion of a polypeptide (e.g., Gal4-AD—at least a portion of HIV integrase), wherein the portion of HIV integrase includes at least SEQ ID NO:9, SEQ ID NO:10, SEQ ID NO:11, SEQ ID NO:12, or any variant thereof having at least east 65%, 70%, 75%, 80%, 85%, 90%, 95%, or 99% sequence homology to SEQ ID NO:9, SEQ ID NO:10, SEQ ID NO:11, or SEQ ID NO:12.

(2) Tripartite Constructs

As described herein, the controlled release hybrid system includes at least one tripartite construct. In certain embodiments, the tripartite construct can include a DNA Binding Domain (DBD) or activation domain (AD); and an interacting domain (e.g., a bait or prey peptide); and a ligand binding domain (LBD). For example, the tripartite construct can have one of the following formula: DBD-LBD-bait or AD-LBD-prey.

In preferred embodiments, the tripartite construct includes, but is not limited to, a sequence encoding for GAL4^{DBD}-LBD-bait or GAL4^{AD}-LBD-prey. For example, in this embodiment, the tripartite construct can be GAL4^{DBD}-LBD-HIV integrase (or a fragment thereof) or GAL4^{DBD}-LBD-HIV integrase inhibiting peptide.

In some embodiments, the tripartite constructs has the formula DBD-PR^{LBD}-bait or AD-PR^{LBD}-prey. In other embodiments, the tripartite constructs has the formula DBD-ER^{LBD}-bait or AD-ER^{LBD}-prey. DBD-GR^{LBD}-bait or AD-GR^{LBD}-prey. DBD-ER^{LBD}-bait or AD-ER^{LBD}-prey.

In a further embodiment, the controlled release hybrid system utilizes at least two different tripartite constructs. In this embodiment, the tripartite construct can have the following formula: DBD-LBD-bait and AD-LBD′-prey, wherein LBD is distinct from LBD′.

When two, different tripartite constructs are used, the tripartite constructs include at least two different LBDs. In this regard, the two different LBDs may optionally recognize and bind to two separate regulatory elements; in addition, the two different LBDs recognize and bind to two separate ligands. By administering two separate ligands at differing concentrations and rates, the hybrid proteins expressed from these two separate tripartite constructs can be differentially controlled. Likewise, the interaction or potential interaction between these hybrid proteins expressed from the two separate tripartite constructs can be differentially controlled.

To further demonstrate, in one embodiment, the first tripartite construct can have the formula GAL4^{DBD}-PR^{LBD}-bait and the second tripartite construct can include a sequence encoding for GAL4^{AD}-ER^{LBD}-prey. Upon expressing the hybrid proteins derived from the two respective tripartite constructs within a host cell, a regulatory element binds to both the PR^{LBD} and ER^{LBD} respectively and prevents the hybrid proteins from interacting. Therefore, transcription and subsequent translation of the reporter-gene fails to occur. However, upon addition of both progesterone, which displaces the regulatory element and binds to the PR^{LBD}, and estrogen, which displaces the regulatory element and binds to the ER^{LBD}, the two separate hybrid proteins are capable of interacting, thus activating the detectable reporter-gene.

C. Regulatory Elements

In certain embodiments, the regulatory element interacts with the LBD either cytoplasmically or within the nucleus to modulate availability of a hybrid protein having a LBD. In some embodiments, the regulatory element binds to the at least one hybrid protein and changes the protein conformation of at least one hybrid protein. By changing the protein conformation, the hybrid protein is no longer able to interact with a second hybrid protein; therefore, formation of a potential interacting pair is essentially blocked in the presence of a regulatory element. By blocking this potential interaction, activation and/or transcription of the reporter-gene is effectively prevented.

Regulatory elements can include, but are not limited to, either naturally occurring or synthetic peptides, polypeptides, proteins, hormones, or any combination thereof. In one embodiment, the regulatory element can include, but is not limited to, a heat shock protein such as bacterial heat shock proteins or eukaryotic heat shock proteins. In certain embodiments, these heat shock proteins are capable of binding to the LBDs described above. For example, in one embodiment, the heat shock protein includes at least yeast Hsp90. In this embodiment, the yeast Hsp90 is capable of binding to the LBD (e.g., PR^{LBD}, ER^{LBD},AR^{LBD}, etc.) of the hybrid proteins described above. Upon Hsp90 binding to the LBD of the hybrid protein, the hybrid protein is no longer able to interact with a second hybrid protein; therefore, formation of a potential interacting pair is essentially blocked when Hsp90 is bound to the LBD. Thus, activation and/or transcription of the reporter-gene is effectively prevented. In certain embodiments, the heat shock protein only includes yeast Hsp90.

D. Ligands

In certain embodiments, it is desirable to displace the regulatory element and potentially activate transcription of the reporter gene described above in a controlled manner. In preferred embodiments, the ligand displays a higher binding affinity for the LBD than the regulatory element. Therefore, the ligand is capable of displacing the regulatory element and binding the LBD. If displacing the regulatory element is desired, a ligand capable of displacing the regulatory element can be administered to the host cell. In certain embodiments, the ligand can be directly administered to the cell without the use of an additional polymer carrier or lipid carrier, directly administered to growth media in which the host cell is growing, administered via lipofection, administered via electroporation, administered via microinjection, or any combination thereof. Upon displacing the regulatory element, the ligand binds the LBD thus allowing the hybrid protein to potentially interact with a second hybrid protein and to potentially activate the detectable reporter gene. In certain embodiments, the ligand interacts with the LBD either cytoplasmically or within the nucleus to modulate availability of a hybrid protein having a LBD. Examples of ligands include, but are not limited to, pharmaceutical agents and modulators, including but not limited to antimicrobial agents, anti-tumor agents, nucleic acid binding agents, cytoskeletal active agents, chelating agents, inducers, co-repressors, and agents affecting intracellular trafficking, localization, and protection or degradation. In certain embodiments, the ligand includes, but is not limited to, a small molecule, a peptide, a hormone (e.g., a steroid), or any combination thereof.

In preferred embodiment, the ligand is a steroid hormone. Suitable steroids include, but are not limited to, androgens (e.g., testosterone, estrogen), progestins, progesterone, corticosteroids, anabolic steroids, ecdysteroids, plant sterols, ergosterols, or any combination thereof. In certain embodiments, steroids include, but are not limited to, progesterone, estrogen, testosterone, testosterone (DHT), androstenedione, androstenediol, dehydroepiandrosterone (DHEA), estradiol, hydroxyflutamide, coumestrol, (DES), p-nonylphenol, bisphenol A, nafoxidine, o,p-DDE, clomiphene, ICI164,384, B-sitosterol, methoxychlor, o,p-DDT, o,p-DDD, methyltestosterone and derivatives thereof, fluoxymesterone, oxymethelone, oxandrolone, methenolone acetate, danazole, 5a-androstan-17B-ol-3-one, methandrostenolone, hydroxyecdysone, stanzolol, and derivatives and analogs thereof. In certain embodiments, the steroids do not include insect steroids including, but not limited to, ecdysone and derivatives thereof.

E. Host Cells

In certain embodiments, the host cells in the controlled release systems include, but are not limited to, prokaryotes, lower eukaryotes, or higher eukaryotes. The host cell can include, but is not limited to, an isolated host cell which includes the controlled release hybrid system described above. The isolated host cell can be selected from the group consisting of a bacterial cell, a fungal cell, a yeast cell, a plant cell, an animal cell, and a mammalian cell. The isolated host cell can further include a cultured bacterial cell, fungal cell, yeast cell, plant cell, animal cell, mammalian cell, or any combination thereof. Examples of host cells include, but are not limited to, fungal or yeast species such as Aspergillus Trichoderma, Saccharomyces, Pichia, Candida, Hansenula, or bacterial species such as those in the genera Synechosystis, Synechococcus, Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium and Klebsiella, plant, animal, and mammalian cells. In some embodiments, the host cell is a yeast cell selected from the group consisting of Saccharomyces, a Pichia, and a Candida host cell. In preferred embodiments the host cell is Saccharomyces cerevisiae. In another embodiment, the host cell is a murine cell. In another embodiment, the host cell is a human cell.

Host cells can be transformed or transfected with the constructs, regulatory elements, ligands, or any combination thereof concurrently (co-transformation or co-transfection) or sequentially using techniques known in the art. These techniques can include, but are not limited to, electroporation, microinjection, chemical techniques (e.g., lipofection, use of calcium phosphate, use of cationic polymers), viral infection, particle bombardment, heat shock, or any combination thereof.

In one embodiment, the host cell is Saccharomyces cerevisiae and the constructs, regulatory elements, ligands, or any combination thereof, are transformed sequentially. In this embodiment, the host cell is first transformed with a construct that expresses the reporter gene. Next, the host cell is transformed with the constructs that express the first hybrid protein and the second hybrid protein either sequentially or concurrently. Following transformation of the constructs that express the first hybrid protein and the second hybrid, the host cell is next contacted with or transformed with a ligand. In this example, the host cell can optionally be contacted or transformed with a regulatory element anytime before contacting the host cell or transforming the host cell with the ligand.

In another embodiment, the host cell is Saccharomyces cerevisiae and the constructs, regulatory elements, ligands, or any combination thereof are transformed sequentially. However, in this embodiment, the host cell is transformed with the constructs that express the first hybrid protein and the second hybrid protein either sequentially or concurrently. Next, the host cell is transformed with the construct that expresses the reporter gene. Following transformation of the constructs that expresses the reporter gene, the host cell is next contacted with or transformed with a ligand. In this example, the host cell can optionally be contacted or transformed with a regulatory element anytime before contacting the host cell or transfecting the host cell with the ligand.

In another embodiment, the host cell is Saccharomyces cerevisiae and the host cell is transformed concurrently with the constructs, regulatory elements, ligands, or any combination thereof.

F. Controlled Release of at Least One-Hybrid Protein

A unique feature of the controlled release hybrid systems includes the continuous adjustability of the detectable reporter gene. In particular, the sensitivity of the detectable reporter gene in the host cell is continuously adjustable by controlling the availability, accessibility, or the interaction between the first hybrid protein and the second hybrid protein. To clarify, by controlling the interaction between the first and second hybrid protein in a continuous manner, reporter-gene expression may also be controlled in a continuous manner. In this embodiment, the controlled release hybrid systems have the capacity to regulate the absolute or relative availability of the first hybrid protein, second hybrid protein, or a combination thereof, and this in turn may ultimately regulate reporter-gene expression.

In certain embodiments, this continuous adjustability is tightly regulated by administering to or contacting the host cell with a specific amount of a ligand. For example, in certain embodiments, the host cell is a yeast cell. In this embodiment, the ligand is administered while the host cell (i.e., the yeast cell) is preferably in mid-log or logarithmic growth phase. The amount of ligand that can be administered to the host cell varies. For example, if the ligand is a steroid, the amount of steroid administered to the host cell varies depending on the K_{D} value of the steroid for the particular LBD; however, the proper amount of ligand can be easily determined using techniques known in the art. In certain embodiments, the K_{D} (i.e., the affinity of the ligand binding to the LBD) must be in range of LBD that is being expressed by the construct(s) described herein. The K_{D} can be readily determined using techniques known in the art. For example, without wishing to be bound by theory, if the LBD is a PR^{LBD}, progesterone can be selected as the ligand, and in certain embodiments, at least 0.5, 1, 2, 3, 4, 5, 6, 7, 8, 9, or nanomolar of progesterone will be used. Likewise, if the LBD is a testosterone-LBD, testosterone can be selected as the ligand, and in certain embodiments, at least 0.5, 1, 2, 3, 4, 5, 6, 7, 8, 9, or 10 micromolar of testosterone will be used. In certain embodiments, if the LBD is an ER^{LBD}, estrogen can be selected as the ligand, and in certain embodiments, at least 0.5, 1, 2, 3, 4, 5, 6, 7, 8, 9, or 10 picomolar of estrogen can be used. Suitable concentrations of ligand can be determined empirically, e.g., by tittering the ligand and measuring its effect on reporter gene expression.

As described above, various host cells, constructs, regulatory elements, and ligands can be used in the controlled release hybrid systems. In one embodiment, the host cell is Saccharomyces cerevisiae having a reporter-gene construct, at least one bipartite construct capable of expressing a first hybrid protein, and at least one tripartite construct capable of expressing a second hybrid protein, wherein reporter-gene expression occurs upon expression and interaction of the first and second hybrid protein. In this example, the at least one tripartite construct is capable of expressing a LBD, wherein the LBD is a Nuclear Receptor LBD capable of binding at least one heat shock protein (Hsp) (i.e., a regulatory element). Upon binding the at least one Hsp, the second hybrid protein having the LBD is sequestered and prevented from interacting with the first hybrid protein. In this embodiment, the second hybrid protein having the LBD can be sequestered in the host cell's cytoplasm while the first hybrid protein is located within the nucleus. Furthermore, the first hybrid protein is preferentially bound to a nucleic acid sequence upstream of the reporter gene's promoter. To induce expression of the reporter gene, the second hybrid protein must translocate from the cytoplasm into the nucleus and interact with the first hybrid protein. In some embodiments, to facilitate translocation of the second hybrid protein from the cytoplasm to the nucleus, a ligand which displays an affinity for the nuclear receptor LBD is administered to the host cell. In this embodiment, the ligand is preferably a steroid hormone. In this example, administration of the ligand (i.e., varied amounts and varied time intervals of ligand administration) allows for a continuous, controlled release of the second hybrid protein and subsequently a continuous, controlled expression of the reporter gene.

In a further embodiment, the host cell is Saccharomyces cerevisiae having a reporter gene construct, at least one bipartite construct capable of expressing a first hybrid protein, and at least one tripartite construct capable of expressing a second hybrid protein, wherein reporter-gene expression occurs upon expression and interaction of the first and second hybrid protein. In this example, the bipartite construct expresses a first hybrid protein having a DBD and a peptide inhibitor of HIV, and the tripartite construct expresses a second hybrid protein having an AD, a Nuclear Receptor LBD (more specifically a PR^{LBD}), and at least a portion of HIV integrase. In this example, upon expressing the second hybrid protein in the host cell, the PR^{LBD} binds a regulatory element, wherein the regulatory element includes at least one heat shock protein (Hsp). Upon binding the at least one Hsp, the second hybrid protein having the PR^{LBD} is sequestered in the cytoplasm and prevented from interacting with the first hybrid protein. In this embodiment, the first hybrid protein is located within the nucleus. Furthermore, the DBD portion of the first hybrid protein is preferentially bound to a nucleic acid sequence upstream of the reporter gene's promoter. To induce expression of the reporter gene, the second hybrid protein must translocate from the cytoplasm into the nucleus and interact with the first hybrid protein. In some embodiments, to facilitate translocation of the second hybrid protein from the cytoplasm to the nucleus, a ligand which displays an affinity for the nuclear receptor ligand-binding domain is administered to the host cell. In this embodiment, the ligand is at least one steroid, wherein the steroid includes at least progesterone. In this embodiment, administration of progesterone (i.e., varied amounts and varied time intervals of ligand administration) allows for a continuous, controlled release of the second hybrid protein, a controlled interaction between the peptide portion of the first hybrid protein and the HIV integrase portion of the second hybrid protein, and subsequently a continuous, controlled expression of the reporter-gene.

The examples above provide for controlled release of at least one hybrid protein, which subsequently regulates reporter-gene expression. However, in certain embodiments, it is desirable to modify the examples above to allow for the controlled release of at least two hybrid proteins. In this embodiment, the host cell can be selected from any host cell described above. In this embodiment, the host cell has a reporter gene construct, at least a first tripartite construct capable of expressing a first hybrid protein, and at least a second tripartite construct capable of expressing a second hybrid protein, wherein reporter gene expression occurs upon expression and interaction of the first and second hybrid protein. In this example, the first and second tripartite constructs are capable of expressing hybrid proteins having separate LBDs. For example, the first tripartite construct can express a first hybrid protein containing ER^{LBD}, and the second tripartite construct can express a second hybrid protein containing PR^{LBD}. Upon expressing the first and second hybrid proteins within the host cell, ER^{LBD} and PR^{LBD} independently bind at least one Hsp. Upon binding the at least one Hsp, the first and second hybrid proteins are sequestered, and the first and second hybrid proteins are prevented from interacting with each other, which prevents reporter gene expression. To induce expression of the reporter gene, estrogen and progesterone are administered to the host cell. In this embodiments, the first and second hybrid protein are capable of interacting upon administration of estrogen and progesterone, and subsequent reporter-gene expression ensues. In this example, reporter-gene expression can be controlled by the availability of both the first and second hybrid proteins having the two distinct Nuclear Receptor LBDs. In particular, administration of two separate ligands (i.e., varied amounts and varied time intervals of ligand administration) allow for a continuous, controlled release of the second hybrid protein and subsequently a continuous, controlled expression of the reporter-gene.

In certain embodiments, the controlled release hybrid systems can be modified to include a three-hybrid system. In this embodiment, an additional molecule (e.g., a nucleic acid sequence, a protein, a third hybrid protein) may be provided that facilitates or is required for the interaction of the first and second hybrid proteins. In one embodiment, this additional molecule is a protein. The additional molecule effectively stabilizes the interaction between the first and second hybrid protein by forming a connection between the first and second hybrid protein when they otherwise would not interact. In certain embodiments, this additional molecule may also be regulated by regulatory elements, ligands, or a combination thereof as described above.

The controlled release hybrid systems described above can be readily adapted for high throughput formats to rapidly screen and identify hundreds, thousands, or millions of potential interacting pairs. For example, if in one embodiment the controlled system is a controlled release yeast two hybrid adapted for high-throughput screening. In this embodiment, cells (i.e., host cells having the controlled release system) may be grown in a 96 well format, a 384 well format, a 1536, or larger well format and dispensed with a plate filler. Using this format, at least 500, 1,000, 5,000, 10,000, 20,000, 30,000, 40,000, 50,000, 60,000, 70,000, 80,000, 90,000, or 100,000 assays screening for interacting pairs can be conducted in a short period of time, such as every two weeks or less. In one embodiment, a yeast screening robot is used and a lab information system (LIMS) tracks samples and the framework of an Oracle database. True positives (i.e., true interacting pairs) may be recovered at a rate of less than 0.5%, at least 0.5%, less than 1%, at least 1%, at least 2%, or at least 5%. True positives can subsequently be rescreened and may be further evaluated using additional assays that test for molecular interactions.

### III. Screening for Agents that Reduce or Inhibit Interacting Protein Binding

In some instances, it is desirable to screen candidate agents for agents that either reduce or prevent the first hybrid protein and the second hybrid protein from interacting. The controlled release hybrid systems and methods of use thereof can be readily modified to screen for such agents. In some embodiments, these agents include, but are not limited to, peptide(s), small organic compound(s), inorganic compound(s), or any combination thereof that reduces or prevents an interaction between interacting molecules. In certain embodiments, these agents can be selected from peptide libraries, chemical libraries, or a combination thereof. For example, if it is desired to identify a peptide that either prevents or reduces molecular interactions of an interacting pair (e.g., a first hybrid protein and a second hybrid protein), a peptide library can be screened using the compositions and methods herein. In certain embodiments, this peptide library can be a commercially available peptide library or a novel, non-commercially available peptide library. In another example, if it is desired to identify a chemical (i.e., small organic compound or small inorganic compound) that either prevents or reduces molecular interactions of the interacting pair, a chemical library can be screened using the compositions and methods described herein. In certain embodiments, this chemical library can be a commercially available chemical library or a novel, non-commercially available peptide library.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s). Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds. In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

Candidate agents encompass numerous chemical classes, but are most often organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

In some embodiments, the candidate agents are proteins. In some embodiments, the candidate agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and vertebrate proteins, and human proteins.

It is important to note that the agent can be administered to the host cell at any desired time. For example, the agent can be administered to the host cell before the host cell is transformed or transfected with any construct described herein, at the same time the host cell is transformed or transfected with any construct described herein, after the host cell is transformed or transfected with any construct described herein, or any combination thereof (e.g., the agent is initially administered to the host cell before transformation or transfection with any construct described herein and then the agent is administered to the host cell again either during or after transforming or transfecting the host cell with any construct). In one embodiment, the agent is preferably added prior to the ligand.

As alluded to above, it is desirable to screen for agents that either reduce or prevent the first hybrid protein and the second hybrid protein from interacting. In certain embodiments, the controlled release hybrid systems and methods described herein can be modified to identify agents that either reduce or prevent intermolecular binding between interacting proteins by the following:

(a) administering to a host cell containing at least one of the controlled release hybrid systems described herein (i) an agent and (ii) a ligand that is capable of displacing the regulatory element, wherein displacing the regulatory element allows the first hybrid protein to interact with the second hybrid protein;
(b) comparing the host cell of step (a) to a control that lacks the agent, and
(c) determining whether administration of the agent either reduces or prevents an interaction between the first hybrid protein and second hybrid protein. In certain embodiments, the agent is preferably added or contacted with the host cell prior to the ligand.

In certain embodiments, the interacting pair of molecules or the potential interacting pair of molecules can be released to allow for complete binding to one another or for incomplete binding depending on the assay that is being conducted; complete binding or incomplete binding to the interacting pair may be determined using techniques known in the art. When screening for agents that either reduce or inhibit the interaction of the interacting pair, a control screen (i.e., a screen without the agent) can be used to determine the amount of at least one interacting molecule that is being released according to the controlled release hybrid system described herein. In certain embodiments, when screening for agents that either reduce or prevents interaction between the interacting pair, the interacting pair is released at half of its previously determined maximal binding. If output of the reporter is observed, that agent potentially reduces or inhibits interaction of the interacting pair. The amount reduction or inhibition can be determined by constructing dose-response curves. Likewise, the IC₅₀ can be determined by constructing dose-response curves and by using the agent at various concentrations. In certain embodiments, the agent can increase the interaction between the interacting molecules, and this would lead to an increased output of the reporter being utilized in that particular screen.

The following example is provided to demonstrate how the controlled systems described herein can be modified to screen for agents that reduce or inhibit molecular interaction of the interacting pair. In certain embodiments, the controlled release hybrid systems and methods described herein can be modified to identify agents that either reduce or prevent intermolecular binding between interacting proteins by the following:

(a) administering to a host cell, wherein the host cell is Saccharomyces cerevisiae containing at least one of the controlled release hybrid systems described above, (i) an agent, wherein the agent includes a peptide, chemical, or a combination thereof, and (ii) a ligand that is capable of displacing the regulatory element, wherein the ligand is at least one steroid and the regulatory element is at least one heat shock protein, wherein the steroid is capable of displacing the heat shock protein which allows first hybrid protein to interact with a second hybrid protein;

(b) comparing the host cell of step (a) to a control that lacks the agent, and

(c) determining whether administration of the agent either reduces or prevents an interaction between the first hybrid protein and second hybrid protein. In certain embodiments, numerous controls can be utilized. In addition to the control mention above, additional controls can include, but are not limited a construct encoding for a DBD-LBD-AD, which could be used to rule out steroid specific inhibitors. In certain embodiments, the control can include, but is not limited to, a separate interacting pair.

When screening for agents that reduce or inhibit molecular interaction of the interacting pair, the controlled release hybrid systems described above can be readily adapted for high throughput formats to rapidly screen and identify hundreds, thousands, or millions of agents that reduce or inhibit the molecular interaction of interacting pairs. For example, if in one embodiment the controlled system is a controlled release yeast two hybrid adapted for high-throughput screening. In this embodiment, cells (i.e., host cells having the controlled release system) may be grown in a 96 well format, a 384 well format, a 1536, or larger well format and dispensed with a plate filler. Using this format, at least 500, 1,000, 5,000, 10,000, 20,000, 30,000, 40,000, 50,000, 60,000, 70,000, 80,000, 90,000, or 100,000 assays screening for interacting pairs can be conducted in a short period of time, such as every two weeks or less. In one embodiment, a yeast screening robot is used and a lab information system (LIMS) tracks samples and the framework of an Oracle database. True positives (i.e., true interacting pairs) may be recovered at a rate of less than 0.5%, at least 0.5%, less than 1%, at least 1%, at least 2%, or at least 5% and the frequency of large negative effects on yeast growth is low (<10%). True positives (i.e., agents that reduce or prevent molecular interaction of the interacting pair) can subsequently be rescreened and may be further evaluated using additional assays that test for molecular interactions.

### IV. Screening for Mutant Alleles

In prokaryotic and eukaryotic cells, mutations arise spontaneously. In certain embodiments, mutations can be benign; however, in other instances, mutations can be deleterious. For example, certain mutations result in decreased efficacy of therapeutic agents or complete drug resistance (e.g., Methicillin-resistant Staphylococcus Aureus (MRSA), mutated strains of influenza such as H1N1, chemotherapeutic resistant tumors, drug resistant HIV strains, etc.). Therefore, identifying the mutation that gives rise to drug resistance is of great importance.

In general, it is of particular interest to identify mutations that may potentially lead to decreased efficacy of agents (e.g., therapeutic agents), and upon identifying such mutations, it is of great importance to subsequently identify new agents that treat and/or inhibit the effects of these newly identified mutations. In this embodiment, the controlled release hybrid systems described above can be readily modified to identify such mutants, and upon identifying such mutants, the controlled release hybrids systems can be used to screen and identify new agents that the target protein having the newly identified mutations.

In certain embodiments, an interacting pair (e.g., a first hybrid protein and a second hybrid protein as described above and within the Examples) is produced as described above. For example, this interacting pair can include a portion of HIV integrase (i.e., a protein or a portion of the protein) and a peptide inhibitor of HIV integrase, wherein the peptide inhibitor binds to the portion of HIV integrase. In this embodiment, the interaction is confirmed by the activation of a reporter-gene using the compositions and methods discussed above and further described within the examples section.

Upon identifying an interacting pair, naturally occurring mutants of the interacting pair can be identified, or in the alternative, artificial mutants using techniques known in the art (e.g., site directed mutagenesis) can be generated. After identifying or generating a mutant, it is of particular interest to screen for a molecular interaction between the mutant and at least one partner of the previously identified interacting pair to determine whether the interaction still exists. For example, if the previously identified interacting pair is a portion of HIV integrase (i.e., a protein) and a peptide inhibitor which binds to that portion of HIV integrase, it is of particular interest first to identify mutants of that portion of HIV integrase and second to screen to see whether the previously identified peptide inhibitor will bind to the mutant with similar efficacy. This can be accomplished by screening the mutant with the known peptide inhibitor and comparing this interaction to the previously identified interacting pair. In certain embodiments, this can be accomplished by detecting reporter-gene output. In certain embodiments, if reporter gene activation is decreased by 1%, 2%, 3%, 4%, 5%, 10%, 15%, 20%, 25%, 30%, 35%, 40%, 45%, 50%, 55%, 60%, 65%, 70%, 80%, 85%, 90%, 95%, 99% in which any percentage can serve as an endpoint within a desired range when compared to a control (e.g., reporter gene activation of the previously identified/known interacting pair) and in which the preceding percentages can be either absolute or relative amounts when compared to a control, this decreased reporter-gene output indicates a decreased interaction between the mutant and the previously identified inhibitor. This decreased reporter-gene output can in turn correlates with decreased efficacy of the previously identified peptide inhibitor.

To identify new peptides that interact with the mutant, peptide libraries can be used to screen for such an interaction. In certain embodiments, these new peptides can be produced using random mutagenesis, degenerate oligonucleotides, DNA shuffling, site-directed mutagenesis, or any method available in the art suitable for creating a peptide library containing peptides within the desired sequence identity. In certain embodiments, these new peptides can be cloned into the constructs described above.

Upon designing these new constructs and contacting a host cell with these constructs, the molecular interaction between the mutant and peptides cloned into the constructs can be assayed and compared to the previously identified interacting pair. In certain embodiments, it is desirable to identify a molecular interaction between the mutant and peptides cloned into the constructs, wherein reporter gene output is at least 50%, 55%, 60%, 65%, 70%, 75%, 80%, 85%, 90%, 95%, 99%, or 100% similar to the reporter-gene output of a previously identified interacting pair, wherein the percentages can serve as an endpoint within a desired range when compared to a control. Furthermore, the preceding percentages can be either absolute or relative amounts when compared to a control.

In a further embodiment, upon identifying a molecular interaction between the mutant and peptides cloned into the constructs, wherein reporter gene output is at least 50%, 55%, 60%, 65%, 70%, 75%, 80%, 85%, 90%, 95%, 99%, or 100% similar to the reporter-gene output of a previously identified interacting pair, it is desirable to screen for agents that either prevent or disrupt the interaction of the mutant and its interacting partner (i.e., a protein, portions of proteins, polypeptides, portions of polypeptides, peptides). For example, if the interacting pair is a mutant of HIV integrase and a mutant binding peptide, a screen for agents that either prevent or disrupt the interaction of the mutant HIV integrase will be conducted, and if any agents that disrupt or prevent this interaction are identified, these agents can be considered as viable candidates that suppress this particular mutant. In certain embodiments, the agent not only disrupts the interaction between the mutant and its interacting partner, but in theory, the agent can also bind to the mutant and act as a potential chemical inhibitor. In certain embodiments, these agents include, but are not limited to, small organic compounds, small inorganic compounds, or a combination thereof. In a further embodiment, these agents can be derived from commercially available chemical libraries or novel, non-commercially available chemical libraries. If an agent disrupts an interaction between the interacting pair and subsequently binds to the mutant, the agent can be identified as a potential chemical inhibitor for that particular mutant.

In certain embodiments, the principles of allele-specific suppression or allele-restrictive suppression can be applied to the control release hybrid systems described herein in order to detect various reactions. Allele-specific suppression refers to a peptide or an agent that is capable of specifically binding to and in certain embodiments, inhibiting a specific mutant allele/protein. Whereas allele-restrictive suppression refers to a peptide or an agent that is capable of specifically binding to and in certain embodiments, inhibiting a mutant allele/protein, a wild-type allele/protein, multiple mutant alleles/proteins, or any combination thereof.

In some embodiments, the disclosed hybrid systems are used to screen for either allele-specific suppression or allele-restrictive suppression of HIV integrase. In certain embodiments, HIV integrase is mutated by PCR amplification under mutagenic conditions or spontaneous mutants can be identified followed by cloning the in frame mutant integrase into a construct having the GAL4^{AD} domain thus generating a library of clones encoding potentially mutated integrase. In some embodiments, the library is transformed into yeast.

In certain embodiments, a strain containing the GAL4^{DB}-bait (optionally having a LBD) and a URA3 reporter under control of the GAL promoter is crossed to the strain containing the GAL4^{AD} HIV integrase library (optionally having a LBD). In certain embodiments, the cells will next be plated onto 5-fluorooctane acid (5-FOA) plates. In the presence of 5-fluorooctane acid (5-FOA) in the growth media, cells expressing orotidine 5-phosphate decarboxylase will die due to conversion of 5-FOA into 5-fluorouracil, a toxic compound. 5-FOA is an extremely useful reagent for the selection of Ura-cells amid a population of URA+ cells. The selection is effective in transformation and recombination studies where loss of URA3+ is desired.

To test for plasmid linkage, AMP resistance plasmids can be isolated from the URA+ cells and retransformed into a strain containing GAL4^{DB}-bait. In certain embodiments, the integrase fragments are cloned into a new vector, sequenced, and retested. Mutations that result in a decreased interaction between the peptide and integrase can be mapped on the known crystal structure of integrase.

In certain embodiments, resistance assays can be performed using a variety of cells and viruses. For example, viruses can be passaged serially in increasing concentrations of test compound. Following each passage, supernatant virus is collected, titrated, and assayed for drug susceptibility. Cloning and/or sequencing of relevant target genes can be performed to identify resistance engendering mutations. These mutations can then be used to identify suppressors in yeast. Unlike the molecular interaction inhibition assays described herein, the resistance assays are dependent on a positive result of the initial hits.

In certain embodiments, the principle of allele-specific suppression is applied to detect integrase peptide interactions. If a mutant integrase decreases the interaction of a wild type integrase binding peptide, such as peptide with the mutant integrase, the mutant suppressing peptides can be selected which recovers the binding/inhibition of the mutant integrase.

To select for natural mutations, millions of yeast can be screened by plating the strain on synthetic plates lacking histidine with added 3-AT (3-amino triazole). Plasmids from colonies that grow on the selection media can be isolated and retransformed into the base strain to check for plasmid linkage. Clones that pass plasmid linkage analysis can be sequenced to identify mutation in the DNA region encoding the peptide that recapitulates the interaction with a mutant integrase. Peptide mutants can be tested with the wild-type integrase as well as other mutant integrase alleles. All different classes of mutations are possible, allele-specific mutations, allele-restrictive mutations, general increases in affinity or mutants that bind to a different region of integrase. Alternatively, the region encoding the peptide can also be mutated using degenerate oligos, primers or split ligation (random DNA oligo). Upon finding a peptide that binds to the mutated integrase, this interacting pair can be used to screen for agents (e.g., chemicals such as small molecules including small organic and small inorganic molecules) that either displace the peptide or prevent or inhibit the peptide from interacting with the mutant integrase.

In certain embodiments, priority will be given to allele-specific suppressor sets that are unaffected by the compounds isolated in the primary screen. These sets will then go through chemical screening to identify chemicals that inhibit resistance strains to the drug. Other embodiments include applications of variation on the themes disclosed above with the use of drugs that reduce or inhibit the interaction between the peptide and integrase.

When screening for either mutants or agents that interact with mutants, the controlled release hybrid systems can be readily adapted for high throughput formats to rapidly screen and identify hundreds, thousands, or millions of potential interacting pairs. For example, if in one embodiment the controlled system is a controlled release yeast two hybrid adapted for high-throughput screening. In this embodiment, cells (i.e., host cells having the controlled release system) may be grown in a 96 well format, a 384 well format, a 1536, or larger well format and dispensed with a plate filler. Using this format, at least 500, 1,000, 5,000, 10,000, 20,000, 30,000, 40,000, 50,000, 60,000, 70,000, 80,000, 90,000, or 100,000 assays screening for interacting pairs can be conducted in a short period of time, such as every two weeks or less. In one embodiment, a yeast screening robot is used and a lab information system (LIMS) tracks samples and the framework of an Oracle database. True positives (i.e., true interacting pairs) may be recovered at a rate of less than 0.5%, at least 0.5%, less than 1%, at least 1%, at least 2%, or at least 5%. True positives can subsequently be rescreened and may be further evaluated using additional assays that test for molecular interactions.

In certain embodiments the controlled release systems are used as a genetic screening tool to develop a platform for personalized medicine. In addition, the controlled release systems described above provide major technical advantages over the currently known systems. These advantages include, but are not limited to: (a) detecting and analyzing molecular interactions of various strengths, without any prior knowledge of even the range of such interaction strengths; (b) avoidance of biologically non-relevant interactions; (c) the detection of potentially very important but currently systemically undetected interaction (e.g., weak interactions); and (d) the potential for actually quantifying in vivo strength of intermolecular binding, as characteristically defined by dissociation constant (Kd) (Estojak, J., et al. Mol. Cell. Biol. (1995) 15, 5820-5829). The practical implications of these and related advantages, include but are not limited to: (a) substantial acceleration of detecting and analyzing protein molecular interactions; (b) elimination of a large subset of biologically irrelevant but previously detected interactions; (c) detection of biologically important new interactions; (d) the potential for true in vivo estimations and correlations of Kd; (e) substantial enhancement of large-scale commercial screening; (f) substantially improved effectiveness and efficiency of identifying and elucidating cellular pathways, potential drug targets; (g) increased sensitivity and identification of interacting pair inhibitors that would otherwise go undetected using currently known systems, (h) screening for interacting pair mutants and potentially designing patient specific therapies in response to such mutants, and a variety of other scientifically and commercially important applications.

### V. Controlled Release Kit

Another feature of the instant controlled release hybrid systems includes kits to facilitate the use of the compositions and methods disclosed herein. Exemplary kits would include the constructs described herein, the host cells described herein, the ligands described herein, the regulatory elements described herein, the agents described herein, or any combination thereof. Also included would be protocols and/or vector maps for use of the compositions for the particular applications and necessary reagents to carry out the applications. Such reagents can include, but are not limited to, buffers, solvents, media and solutions, substrates and cofactors, vectors, host cells, detection or reporter genes, or any combination thereof. Accessory items may include vials, vessels, reaction chambers and instructions.

### EXAMPLES

The following examples are included to demonstrate various embodiments of the invention. It should be appreciated by those of skill in the art that the techniques disclosed in the examples which follow represent techniques discovered by the inventors to function well in the practice of the invention. However, those of skill in the art should, in light of the present disclosure, appreciate that many changes that can be made in the specific embodiments which are disclosed and still obtain a like or similar result without departing from the spirit and scope of the invention.

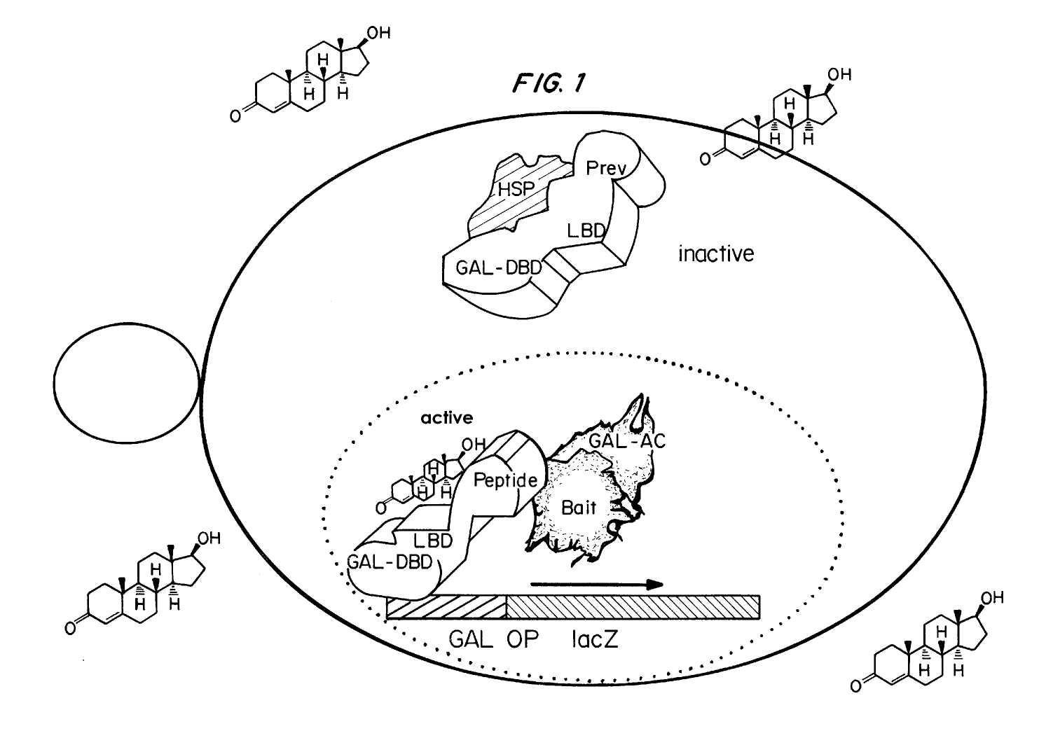

FIG. 1 illustrates a schematic tripartite system according to an embodiment of the present invention. A yeast cell comprises an inactive GAL^{BD}-LBD-Prey in the cytoplasm, which may be bound to a heat shock protein (HSP). Upon binding with a progestin that has crossed the cell membrane, the prey becomes activated and enters the nucleus. In the nucleus, the prey end of the active tripartite system binds to the bait-AD which in turn activates the hybrid system at the binding site or operator (OP) of a lacZ reporter gene. This binding interaction activates transcription of β-galactosidase, which may be monitored via known luminescent detection techniques.

Addition of steroid (e.g., a progestin in FIG. 1) releases the tripartite construct allowing the two protein pairs to interact and activate a reporter. The idea is to add steroid to titrate the amount of release of the protein of interest. Chemicals can be added at the same time as the steroid allowing the chemical to interact with the Bait prior to binding of Prey with the Bait. Alternatively, the regulated release could be coupled to the activation Domain-NHLBD (nuclear hormone ligand binding domain)-Bait. In addition both proteins could be released in different amounts with different timing if Bait and Prey is fused to separate NHLBD under the control of different steroid ligand binding domain e.g. Progesterone and testosterone. This system could be applied to the lex or GAL two-hybrid systems or a mammalian cell system.

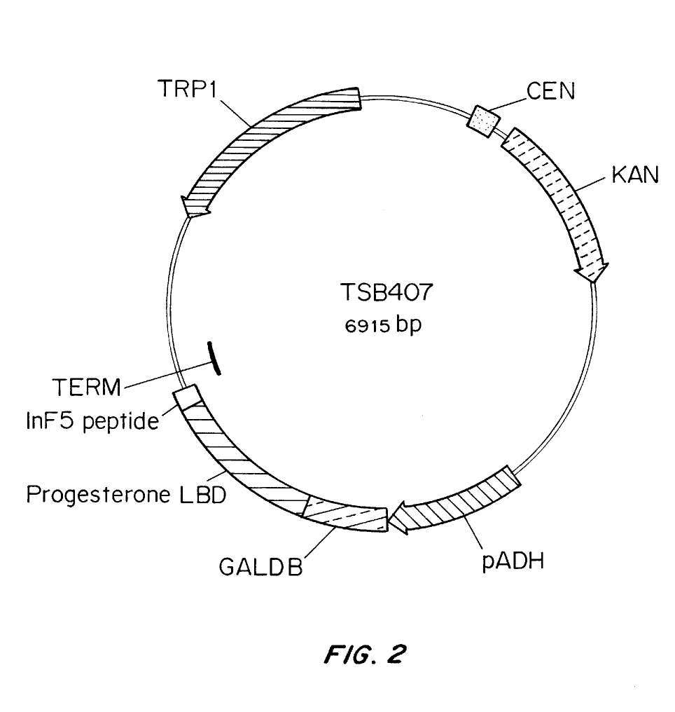

An example of a base plasmid for the steroid two hybrid system is shown in FIG. 2. The plasmid contains the following features: CEN, a yeast selectable marker, a bacterial selectable marker and origin of replication, a promoter driving expression of a fragment of a protein that can bind to a promoter element-protein domain that can regulate the release of the attached protein fragment. In this example, the GAL4 DNA binding domain (GAL^{BD}) is fused to the progesterone nuclear hormone receptor ligand binding domain (PR^{LBD}) and an integrase inhibitory peptide INFS. The chimeric tripartite protein is under the control of the ADH promoter.

To construct the plasmid, the progesterone receptor was amplified using primers oPRLBDF-5′ GCT TTG ACT CAT ATG ATA CAG TTG ATT CCA CCA CTG ATC AAC CTG TTAATG AG (SEQ ID NO:17) and oPRLBDR-5′ CCT CGG CCC ATA TGG GCT TTA TGAAAG AGA AGG GGT TTC ACC ATC CC (SEQ ID NO:18) using image clone 5167591 as template encoding the progesterone receptor (Invitrogen, Carlsbad, Calif.). The PCR fragments encoding the progesterone receptor ligand binding domains were digested with NdeI and ligated into the NdeI site of pVT2114-F5 placing the release of F5 peptide under the control of the addition of a progestin steroid. Optimization of linkers, promoters, activators, codon bias, reporters, and spacing between LBD and interacting molecule for optimum interaction may improve the signal strength without changing the scope of the invention.

### Example 1

### Peptide Library Construction for Steroid Release System

This library can be used for bait's that are activators and can not be placed in the DNA binding domain construct since they will falsely activate in the absence of a interacting partner linked to the activation domain. Briefly, a 15-mer of randomized nucleotides flanked on either side by constant regions were inserted into the Sfi1 site of TSB428 vector (CEN plasmid, Adh-GAL^{BD}-PR^{LBD}-Stuffer, TRP1) using the splint ligation procedures (see, for example, Abedi M. R. et al., (1998) N.A.R. 26(2): 623-630). As a result of these procedures, the peptides were fused to the C-terminus of the GAL^{BD}-PR^{LBD} chimeric protein. Library members of (−3.3×10⁶ in all) can be achieved were then transformed into bacteria (DH5α, Gibco, BRL), grown in liquid culture under selective conditions (e.g. 40 ug KAN/ml), and prepared for transformation into yeast (Qiagen MaxiPrep) to identify peptide binders of Bait proteins. MATa, ura3-52, his3-200, ade2-101, trp1-901, leu2-3, 112, gal4Δ, met⁻, ga180Δ, GAL1_{UAS}-GAL1_{TATA}-URA3 lys2:GAL1_{UAS}-GAL1_{TATA}-lacZ) using standard techniques (LiAc TRAFO Method) and plated on SD, -TRP agar plates. False activators can be removed from the library by using FOA and steroid.

This library can be peptide can be linked to the activation domain. Briefly, a 15-mer of randomized nucleotides flanked on either side by constant regions were inserted into the Sfi1 site of TSB428 vector (CEN plasmid, Adh-GAL^{A}-AR^{LBD}—Stuffer, LEU2) using the splint ligation procedures. As a result of these procedures, the peptides can be fused to the C-terminus of the GAL^{AD}-AR^{LBD} chimeric protein. Library members of (˜3.3×10⁶ in all) can be achieved were then transformed into bacteria (DH5α, Gibco, BRL), grown in liquid culture under selective conditions (e.g. 100 μg AMP/ml), and prepared for transformation into yeast (Qiagen MaxiPrep) to identify peptide binders of Bait proteins. The strain MATα, ura3-52, his3-200, ade2-101, trp1-901, leu2-3, 112, gal4Δ, met⁻, ga180Δ, GAL1_{UAS}-GAL1_{TATA}-URA3 lys2:GAL1_{UAS}-GAL1_{TATA}-lacZ) can be transformed using standard techniques (LiAc TRAFO Method) and plated on SD, -LEU2 agar plates and mated to a strain that contains a plasmid encoding the bait linked to the DNA-binding domain. Using mating for plasmid linkage has been described in Sandrock et al., Genetics (1999) 151: 1287-1297.

### Example 2

### Screening for Peptide Binders Using Steroid Release System

To identify peptides that demonstrate an affinity for the Bait, Mat a, trp1-901, leu2-3, 112, ura3-52, his3-200, gal4Δ, gal 80Δ, LYS2::GAL1_{UAS}-GAL1_{TATA}-HIS3, GAL2_{UAS}-GAL2_{TATA}-ADE2, URA3:MEL1_{UAS}-MEL1_{TATA}′ lacZ)[[pADH-GAL^{BD}:PR^{LBD}:randompep, CEN, TRP1, Kan^{R}] containing the random peptide library was grown in liquid media (SD-Tip), and then mated to Mat alpha, trp1-901, leu2-3, 112, ura3-52, his3-200, gal4Δ, gal 80Δ, LYS2::GAL1_{UAS}-GAL1_{TATA}-HIS3, GAL2_{UAS}-GAL2_{TATA}-ADE2, URA3:MEL1_{UAS}-MEL1_{TATA}-lacZ) [pADH-GAL^{AD}-Bait, CEN, LEU2, Amp^{R}] expressing the target plasmid.

Briefly, this was accomplished by mixing 4×10⁷ log-phase GAL4^{BD}—NHR-peplibrary cells with 2×10⁷ log phase cells containing the pADH-GAL^{AC}-Bait, CEN, LEU2, Amp^{R} in YEPD at a density of 10⁶ cells per ml. The culture, containing both “a” and “alpha” mating types, was then allowed to sit at room temperature (no shaking) for twenty-four hours before being centrifuged, resuspended in a small volume, and plated out on Leu⁻Trp⁻ minimal media plates (48 hrs, 30° C.) to select for diploids. The diploid colonies (Leu⁺Trp⁺) were subsequently pooled and re-plated on selective dropout plates designed to identify peptide binders (Leu⁻, Trp⁻, +steroid, His⁻+2.5 mM 3-AT). 3.79×10⁸ diploids were plated on selection plates. Individual colonies were then patched on selective media. Plasmid DNA was isolated using (YDER) and transformed into E. coli. Mini-prepped DNA was re-transformed back in for plasmid linkage. The interaction can also be tested in the presence or absence of steroid. The key is that the chimeric bait and prey can be unrelated to nuclear hormone receptors.

### Example 3

### Steroid Release Two Hybrid Interaction

To test the two hybrid release system, plasmids and were transformed into base strain TSY 201 MATa ura3-52, his3-200, ade2-201, lys2-801, trp1-901, pdr5-delta, sng2-delta, leu2-3, 112, ga14-542, ga180-538 LYS2::GAL2_{UAS}-GAL1_{TATA}-HIS3, URA3::GAL4_{17mers(X3)-}CyC1_{TATA}-lacZ. The strain contains integrated LacZ and HIS3 reporters for the detection of two-hybrid interactions.

A positive control (GAL4^{BD}-PR^{LBD}-GAL4^{AD}) is highly sensitive to progesterone levels and does not require interaction to activate the reporter and shows that the yeast system and conditions can activate the PRLBD.

Wide ranges of steroids concentrations in the yeast medium result in wide ranges of variable and relative release of the hybrid proteins. This results in variable release of the bait first hybrid protein over a continuous range of amounts in response to changing levels of progesterone in the yeast growth medium. A control yeast strain (not shown) containing TSB 407 expressing (GAL4^{BD}-PR^{LBD}-INF5) plus no-interacting protein pVT 702 (GAL4^{AD} control) resulted in no increase in expression with increased addition of steroids. The yeast strain containing the TSB400 and TSB413

GALAC fused to HIV integrase (1-210 a.a.) showed an increase in signal over a wide range of progesterone concentrations. In the presence of progesterone, the RFU increases only in the strain expressing integrase fused to the GAL4^{AD} and the interacting peptide (GAL4^{BD}-PR^{LBD}-IN5). The positive control (GAL4^{BD}-PR^{LBD}-GAL4^{AD}) was also responsive to steroid

Yeast LacZ assays were performed as follows. Yeast strains were grown to saturation by overnight incubation with shaking at 30° C. in 50 ml of selective media auxotrophic for tryptophan and leucine (supplemented with 2% dextrose). The yeast culture were then be diluted in selective media (supplemented with 2% dextrose) to a final OD600 absorbance value of 0.01-0.05. Sixty microliters of this cell suspension were aliquoted into each well of a 384-well microplate (CliniPlate #11310-888, Thermolabsystems, Fi). Progesterone at various concentrations (or neat DMSO in the case of the controls) were added onto the yeast in each well of the 384-well microplate. The 384-well microplates containing yeast plus chemicals were be grown in a Sterile Cult incubator for 18 hours. Following the incubation in the incubator, cells were resuspended and the OD600 absorbance value of each well was measured and recorded using a Saphire II microplate reader (Molecular Devices). Nine microliters of cells per-well were then be transferred into a black 384-well microplate (Matrical low volume MP101-1-PS, Spokane Wash.). Three microliters of YPER Plus (Pierce Chemicals) were added to each well (containing yeast). The plates were incubated at room temperature for 30 minutes in order to allow for yeast lysis. 22.5 μl of 1 mM CUG (β-galactosidase substrate, Molecular Probes) in Z-buffer (60 mM Na₂HPO₄.7H₂O, 40 mM NaH₂PO₄.H₂O, 10 mM KCl, 50 mM MgSO₄.7H₂O, 50 mM β-mercaptoethanol) was then added to each well. The solution was incubated for 30 minutes at room temperature following which 2.5 μl of 1M Na₂CO₃, added to stop the reaction. The fluorescence value of each well was then be measured and recorded using a Sapphire II reader (Tecan) with excitation at 390 nm and emission recorded at 460 nm with a 455 nm cutoff filter. Both the OD600 absorbance values and the fluorescence values will be subtracted for background and a normalized fluorescence value will be obtained by dividing the fluorescence value (for any given well) by the OD600 absorbance value (of the corresponding well).

### Example 4

### Chemical Screening Using the Steroid Release Two Hybrid Interaction

The yeast two hybrid approach and variants thereof have been widely used in recent times for a variety of investigations into protein protein interactions. This example illustrates a yeast base controlled release two hybrid screening platform suitable for screening individual chemical compounds. The system is based on the principle that a transcriptional reporter can serve as a surrogate to measure the degree of complex formation between a target, such as a particular enzyme or any large protein and a peptide which binds to the target. Chemicals that interfere with the interaction between peptide and target will reduce reporter transcription. Experiments were performed to optimize several parameters of this variant of the yeast two hybrid system. Based on these experiments, the enzymatic reporter (LacZ), CEN plasmids, and mutant strains of yeast which lack some elements of multi-drug resistant (MDR).

Yeast Strains Suitable for Chemical Screening

Yeast cells are notoriously responsive to keeping external agents out and removing them once inside so in order to increase drug penetration levels into the cells, we engineered yeast strains which were defective in MDR-type efflux pumps. These strains alleviate one potential problem for in vivo drug screening in yeast based systems: the active transport of drugs out of the cell. Yeast has several genes encoding ATP-binding cassette transporters (ABC) proteins involved in the MDR phenotype. The ABC proteins are generally able to reduce the intracellular concentrations of exogenous chemicals as they transit into the cells. Targeted deletions were made in two major genes encoding these ABC transporters: PDR5, and SNQ2. The genotypes of the strain constructed are TSY 201 MATa ura3-52, his 3-200, ade2-201, lys2-801, trp1-901, pdr5delta, sng2delta, leu2-3, 112, ga14-542, ga180-538 LYS2::GAL2_{UAS}-GAL1_{TATA}-HIS3, URA3::GAL_{407mers(X3)-}CyC1_{TATA}-lacZ. The strain was co-transformed with plasmids, pVT407 (CEN, Adh-GAL^{BD}-NHPR-pep, TRP1) and TSB413 (CEN, Adh-GAL^{AD}-IN, LEU2).

Chemical Screening Platform

This system is based on the principle that a trancriptional reporter can serve as a surrogate to measure the degree of complex formation between interacting molecules. Chemicals that interfere with the interaction or binding of the interacting molecules will reduce transcription. In particular, this example demonstrates a controlled release two hybrid system adapted to screen individual compounds that potentially reduce or inhibit the interaction of interacting molecules. As described in the materials section below, integrase (1-210 aa) and the peptide LYETILILLFLDVDT (SEQ ID NO:7) serve as the interacting molecules. Furthermore, progesterone is used as the ligand that facilitates controlled release.

Materials:

Cell culture media and related materials were as follows: CM Glucose Broth minus LEU, TRP with ADE-0.1 (Teknova); AeraSeal Breathable sealing film (ISCBioexpress). The yeast strains were kept at −80 degrees C. until use.

The yeast screening strain was MATa ura3-52, his3-200, ade2-201, lys2-801, trp1-901, PDR5delta, SNQ2delta, leu2-3, 112, ga14-542, ga180-538 LYS2::GAL2_{UAS}-GAL1_{TATA}-HIS3, URA3::GAL4_{17mers(X3)-}CyC1_{TATA}-lacZ harboring pBAL26 (pGALAC-IN (1-210 aa), LEU2, AMP) and TSB 408 (pGAL^{BD}-PRNHR-SEQ ID NO:7, TRP1, KAN).

The yeast control screening strain MATa ura3-52, his3-200, ade2-201, lys2-801, trp1-901, PDR5delta, SNQ2delta, leu2-3, 112, ga14-542, ga180-538 LYS2::GAL2_{UAS}-GAL1_{TATA}-HIS3, URA3::GAL⁴_{17mers(X3)-}CyC1_{TATA}-lacZ TSB410 (GAL^{BD}-PRNHR-GAL^{AD}, TRP1, KAN) and (pVT3210 pGAL^{AD}, LEU2, AMP). PRNHR encode the progesterone hormone binding domain. Progesterone (catalog #P3972) was from Sigma. Beta-Glo assay kits (catalog #E4780) were from Promega. Assay plates (384 well, clear bottom, white tissue culture plates) were from BD Falcon (catalog #353963).

Methods

For the primary screen and confirmation assays, an overnight culture of the yeast screening strain was prepared by quickly thawing the vial of yeast and immediately adding 150 μl of culture into a sterile Ehrlenmeyer flask containing 50 ml of media and shaking at 30° C. at 250 rpm. Vials of yeast culture were subjected to less than 4 freeze/thaw cycles. The overnight culture was diluted to an OD600 of ˜0.03. Assay plates were prepared by dispensing 150nL/well of 1 mM compound in 100% DMSO (PlateMatePlus positive displacement/fixed tip head, Thermo) and then dispensing 30 μl of diluted culture suspension using a Matrix WellMate equipped with a microbore manifold. The yeast suspension was agitated frequently during addition to prevent the cells from settling. The final compound concentration was 5 μM. Plates were incubated for 1 hour at 30° C. in a humidified incubator. Progesterone (200 nl/well in 50% DMSO/media) was then added using a Thermo CombinL dispenser. Final progesterone concentration was 8 μM and final DMSO concentration was 0.88%. Plates were sealed with an Aeraseal cover to minimize evaporation and incubated for 17-22 hours at 30° C. in a humidified incubator. The Aeraseals were carefully removed and 30 μL/well of room temperature Beta-Glo (Promega) reagent was added to each well using a dedicated Matrix microbore manifold, rotated in a circular motion on a plate rotator for 10 seconds and then incubated for 1-2 hours in the humidified incubator. Luminescence was read on a BMG Pherastar plate reader.

For the primary screen, each plate contained 24 uninhibited control (DMSO only) wells and 8 “inhibited” control wells (no progesterone). For dose response assays, compounds were tested at 11 concentrations, from 25 to 0.025 μM. 5 mM compound in 100% DMSO was prepared and diluted the compound 1:2 in 100% DMSO (11 dilutions). Next 0.15 μl of each dilution was dispensed to the assay plate before adding 30 μl yeast culture. For the dose response assays, the OD610 was measured for the plates before adding the Beta-Glo reagent. Active compounds were also tested in dose response assays against a second yeast strain. This strain was responsive to progesterone, but did not contain the HIV integrase target. Progesterone concentration used for this strain was 0.1 μM. IC₅₀ values were calculated using XLFit (IDBS, Inc.).

Assay Validation

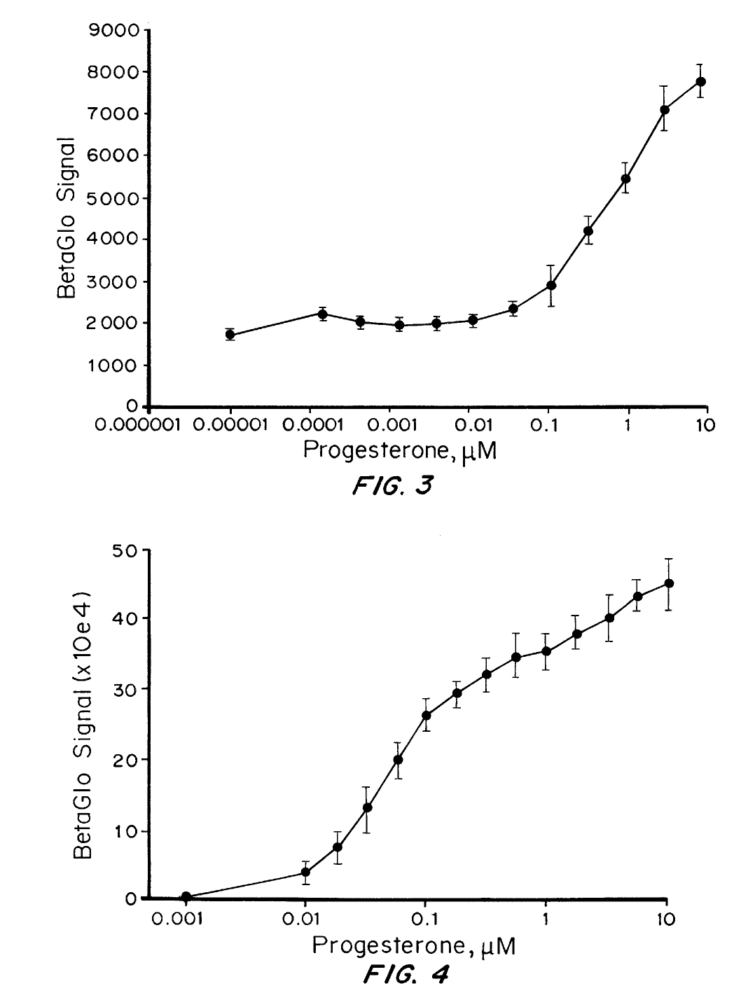

The response of the yeast screening strain to progesterone was evaluated. FIG. 3 shows that progesterone stimulated expression of beta-galactosidase. The signal-to-background at 8 μM progesterone was ˜4 with good reproducibility (% CV=8%). 8 μM was used to stimulate the screening strain in all experiments.

A control strain (responsive to progesterone, but lacking the HIV integrase target) was also evaluated FIG. 4. This strain was much more sensitive to progesterone, and provided a much greater response. The signal-to-background was ˜100. For compound evaluation, the control strain was stimulated with 0.1 μM progesterone.

Primary Screen

The MHTSC Select Set was screened at a concentration of 5 μM. Control data suggested that the assay was consistent and robust, with a good separation between the progesterone stimulated and unstimulated wells. The signal-to background was consistently ≧2.5.

Control values were used to calculate the Z′ value using the equation: Z′=1-((3×(SDuninhib.+SDinhib))/(Meanuninhib−Meaninhib)). Z′ values are commonly used to describe the quality of a screening assay. Values >0.5 are considered acceptable for a good screening assay (Zhang et al. (1999) J. Biomol. Screen. 4, 67-73). Z′ values, were all ≧0.4, suggesting that the assay was reasonably reproducible.

The variation of the uninhibited control values was used to set the cut-off for the active compounds. Compounds with luminescence values less than the average stimulated control minus 3 times the standard deviation of the stimulated control were considered active. This cut-off represents 26-49% inhibition (depending on the variability of the data on each plate). 255 hits were identified for further study.

Confirmation Assays

Confirmation assays were conducted on 255 active compounds at the same (5 μM) concentration in quadruplicate. The cut-off for activity (for each replicate) was 3× the SD of the stimulated control. Compounds were considered confirmed active if they were active in at least 3 of the 4 replicates. 203 compounds were confirmed active, and further evaluated in dose response assays.

Dose Response Assays

Dose response assays were conducted on the 203 confirmed active compounds. Compounds were evaluated at 11 concentrations from 25 to 0.025 μM. Before beta-galactosidase activity was measured, OD610 was read as a measure of toxicity.

The four parameter logistic equation was utilized in determining IC₅₀'s and Hill Slopes of compounds. Curve fitting was conducted by two methods. First, the curves were fit using the data for the 11 concentrations for each individual compound. Then the minimum and maximum values were determined from that data. The term “float” is used to characterize this method, since the calculated minimum and maximum “float” based on the data. In the second method, the minimum and maximum are “fixed” based on stimulated and un-stimulated control wells. This allows us to calculate IC₅₀ values for compounds that do not pass the inflection point. Usually, these are weakly active compounds with IC₅₀s greater than the highest concentration tested. Weakly active compounds only show activity at the highest concentrations and will not reach a “plateau” minimum value. If the curve fit is allowed to “float” for these compounds, the calculated minimum value will be much less than the actual minimum and the calculated IC₅₀ will be incorrect. Using the same fixed maximum and minimum also standardizes the curves to allow better comparisons between compounds. Ideally, the “float” and “fixed” values will be very similar. When the “fixed” data was considered, 101 compounds produced well-behaved dose response curves (Hill Slopes between 0.4 and 3) with IC₅₀ values<50 μM.

Data from OD₆₁₀ measurements was used to determine potential compound cytotoxicity. There were 20 compounds that appeared to inhibit cell growth by at least 50% at 25 μM (the highest concentration tested). This suggests that there were 81 compounds that inhibited beta-galactosidase without toxicity.

The control strain was used to identify compounds that inhibited beta-galactosidase in the absence of the HIV integrase target. These compounds could directly inhibit beta-galactosidase activity or could interfere with the progesterone stimulation. Most of the compounds inhibited the control strain. 16 compounds were identified that had IC50 values for the HIV containing strain that were less than half of the IC50 for the control strain. These are the potential “selective” inhibitors of the HIV target.

A high throughput screening assay for the interaction of HIV integrase and an integrase-specific peptide using a modified two-hybrid assay in yeast was validated and implemented. The two hybrid system required a steroid (progesterone) as well as protein-peptide interaction in order to activate a reporter gene (beta-galactosidase). The progesterone concentration was optimized to generate a robust and reproducible signal. The MHTSC Select Set diversity library and the MHTSC Known Inhibitor library were screened for inhibition of HIV integrase-peptide binding. The assay was robust, with a signal-to-background >2.5. Compounds were tested at 5 μM in the primary screen. 255 hits were identified using a cutoff of 3× the standard deviation of the uninhibited controls. Confirmation assays, conducted in quadruplicate at 5 μM, gave 203 active compounds. These 203 compounds were tested in dose response assays, from 25 to 0.025 μM against two strains of yeast—the screening strain containing the integrase-peptide constructs and a control strain that did not contain the target, but was responsive to progesterone. The control strain was evaluated to eliminate compounds that inhibited progesterone binding. Of these 203 compounds, there were a small number (10-15) that appeared to be selective for the integrase-peptide dependent strain. IC₅₀ values for the selective compounds ranged from 0.5 to 20 μM.

### Example 5

### Screening Mutants of Interacting Molecules

Allele-specific suppressors can be identified that restore the binding to the mutant integrase. The yeast strains and plasmids with resistance characteristics for each protein-peptide can be used as described below to screen chemical libraries in a high throughput manner.

Allele-specific suppression has been used extensively as a genetic tool for the identification and analysis of physical interactions involving RNA, DNA, and protein. A widely held view of the mechanism by which allele-specific suppression occurs invokes the “lock-and-key” model, in which the original contact is restored. In the case of RNA-RNA interactions, interactions can be restored by mutations that allow compensatory changes in base-pairing. Allele-specific suppression is often taken as evidence of protein-protein interactions; conversely non-allele specific suppression is usually taken as evidence of bypass suppression.

Peptides that bind to the core domain of HIV integrase were previously determined. Integrase can be mutated by PCR amplification under mutagenic conditions followed by cloning the PCR product encoding integrase in frame with the GAL4^{AD} domain generating a library of clones encoding potentially mutated integrase. The library can be transformed into yeast.

A strain containing the GAL4^{BD}-bait peptide and a URA3 reporter under control of the GAL promoter can be crossed to the strain containing the GAL4^{AD} HIV integrase library. The cells can be plated onto 5-fluorooctane acid (5-FOA) plates. In the presence of 5-fluorooctane acid (5-FOA) in the growth media, cells expressing orotidine 5-phosphate decarboxylase can die due to convertion of 5-FOA into 5-fluorouracil, a toxic compound. 5-FOA is an extremely useful reagent for the selection of Ura-cells amid a population of URA+ cells. The selection is effective in transformation and recombination studies where loss of URA3+is desired.

To test for plasmid linkage, AMP resistance plasmids can be isolated from the URA+ cells and retransformed into a strain containing GAL4^{BD}-bait peptide. The integrase fragments can be cloned into a new vector, sequenced, and retested. Mutations that result in the decrease in interaction between the peptide and integrase can be mapped on the known crystal structure of integrase.

The resistance assays can be performed using a variety of cells and viruses. Virus is passaged serially in increasing concentrations of test compound. Following each passage, supernatant virus is collected, titrated, and assayed for drug susceptibility. Cloning and/or sequencing of relevant target genes can be performed to identify resistance engendering mutations. These mutations can then be used to identify suppressors in yeast. Unlike the yeast genetic approach, the resistance assays are dependent on a positive result of the initial hits; starting with a small molecule hit.

The principle of allele-specific suppression can be applied to detect integrase peptide interactions. If a mutant integrase decreases the interaction of a wild type integrase binding peptide, such as peptide with the mutant integrase, the mutant suppressing peptides can be selected which recovers the binding/inhibition of the mutant integrase.

To select for natural mutations, millions of yeast can be screened by plating the strain on synthetic plates lacking histidine with added 3-AT (3-amino triazole). Plasmids from colonies that grow on the selection media can be isolated and retransformed into the base strain to check for plasmid linkage. Clones that pass plasmid linkage analysis can be sequenced to identify mutation in the DNA region encoding the peptide that recapitulates the interaction with a mutant integrase. The peptide mutants can be tested with the wild-type integrase as well as other mutant integrase alleles. All different classes of mutations are possible, allele-specific mutations, general increases in affinity or mutants that bind to a different region of integrase. Alternatively, the region encoding the peptide can also be mutated using degenerate oligos, primers or split ligation (random DNA oligo). Once finding a peptide that binds to the mutated integrase, it can be used to screen for chemicals that displace the peptide.

Priority can be given to allele-specific suppressor sets that are un-affected by the compounds isolated in the primary screen. These sets can then go through chemical screening to identify chemicals that would inhibit resistance strains to the drug.

Upon identifying integrase mutants, the integrase mutations can be mapped onto the crystal structure of integrase. Allele-specific suppressors may give some more insight into the design of potential drugs and future diagnostic tests.

TABLE 1
Allele Specific Suppression

| Genotype | | Phenotype Chemical Inhibitor | | Future Chemical Screens |
|---|---|---|---|---|
| IN+ | pep+ | Growth | Low | Yes |
| *IN+ | pep 1-1 | Growth | High | Yes |
| IN+ | pep 1-1 | | NA | No |
| ln 1-1 | pep 1-1 | Growth | High | Yes |
| ln 1-1 | pep+ | | NA | No |
| ln 1-2 | pep+ | Growth | High | Maybe |
| ln 1-2 | pep 1-1 | | NA | No |

*Could represent a general increase in affinity of the peptide for integrase
Growth = interaction
Low = disrupts interaction between peptide and integrase
High = chemical does NOT disrupt interaction
NA = not applicable, cannot be tested because no interaction

Allele-specific suppression would allow interaction with specific alleles of integrase but not all alleles. Interaction could be detected through an auxotrophic reporter or LacZ reporter. Priority can be given to allele-specific suppressor sets that are un-affected by inhibitory compounds (see Table 1). These sets can then go through chemical screening.

Chemical Screening of Mutant Interacting Molecules

Allele-specific suppressor sets that are un-affected by previously isolated compounds can be used in chemical screening as described below. To date, it appears that no one has used allele-specific suppression for HIV integrase in yeast to feed assays into chemical screening.

Utilizing the novel steroid release two hybrid yeast system described herein, a chemical screening platform can be utilized to identify chemical inhibitors of HIV integrase mutants. The system is based on the principle that a transcriptional reporter can serve as a surrogate for the degree of complex formation between a target such as HIV integrase and a peptide inhibitor of the target. Chemicals that interfere with the interaction between peptide and target can reduce reporter transcription.

### Example 6

### Transfer of Peptides Under Control of NHLBD for Analysis of Phenotypes in Mammalian Cells

Peptides isolated through this technology can be tested for phenotypes in mammalian cells using the protocols below. The phenotype in the mammalian system can also be controlled by the release of the peptide in the mammalian cell line. For example, methods for identifying agents that alter a phenotype of interest, such as those described in U.S. Pat. Nos. 5,955,275, 5,998,136, 6,025,485, 6,566,057, 6,579,675, 6,582,899, and U.S. Patent Publication Nos. US2002/0019005, US2002/0045188, US2003/0054389, WO2004/012574, WO2003/008648, which are incorporated by reference in their entirety for description of these compositions and methods for use with the peptides isolated using the disclosed technology.

Plasmid Constructions

Retroviral constructs used to express the ZsGreen (Zoanthus green fluorescent protein, Clontech) scaffold, the Zsgreen-PepA and Zsgreen-PepB constructs from various promoters were constructed in the following manner. pVT1614 a MMLV(Molony murine leukemia virus) retroviral vector which contains the Zsgreen protein expressed from the HIV2 promoter was digested with Sfi1 (New England Biolabs, NEB) and ligated to Sfi1 fragments encoding either a stop codon, PepA and PepB creating respectively. The expression fragment in the retroviral vectors can be LBD-peptide. Numerous retroviral expression vectors can be used with different reporters etc. to fine tune the expression to the cell line of interest.

Cell Culture Methodology

Retroviral packaging, cellular transductions, cell culture and the sytox-based cytotoxicity assay were carried out. Briefly, DNAs encoding the various retroviral constructs were arrayed in 96-well plates and subsequently packaged, in a 96-well format in 293gp cells using an automated 96-channel pipettor (Beckman Multimek, Beckman Coulter Instruments; Fullerton, Calif.). Supernatants were then used to transduce the various cell types (50% v/v) that had been seeded one day prior to transduction in clear/flat-bottomed 96-well plates (Greiner). Cells were seeded at the following densities; HT29 and SW620, 2000 cells/well, HeLa cells 400 cells/well, WI-38, HuVEC and A549 cells 600 cells/well PrEC cells 700 cells/well. Cells were grown in the following media at 37° C. and 5% CO₂. HT29, SW620, A549, HeLa, WI-38 in DMEM (Gibco) supplemented with 10% FBS and 2 mM L-glutamine. PrEC in PREGM media (PREBM supplemented with, Clonetics). HuVEC in EGM-2 (EGBM+. Retroviral supernatants were removed via a media change ˜24 hrs. post transduction. Four days post-transduction, Sytox Orange (Molecular Probes, Eugene Oreg.) was added to each well of the assay plate to a final concentration of 1 μM. The plates were allowed to incubate for 20 minutes at 37° C., then the fluorescence read on a CCD imaging system (Ex: 475+/−15 nm, Em: 515+/−10 nm). After reading, cells were permeabilized with saponin (with the following exceptions) by adding it to each well at a final concentration of 0.1%, mixing, and incubating for approximately 2 hours at 37° C. The fluorescence of each well was then read again on the CCD. HuVEC and PrEC cells were permeabilized using X % tween-20 and WI-38 by freeze-thawing cells in a −80° C. freezer for 15′. Fluorescent data analysis, specifically the number of fluorescent cells and the total number of bright pixels/well was performed using software written in-house.

Somata Assay

To begin the bioassay, supernatants of retroviruses encoding the binding peptide(s) were transduced into mammalian cells (ATCC # CCL-1958) that express the target of the peptide. Transductions into mammalian cells were performed by plating 500-1200 cells in microtiter plate wells in a total volume of 100 ml media and allowing cells to attach over the course of several hours. After transduction, cells were cultured for an additional 4-5 days before determining the effects of the Bait-binding peptide. The affects could be tested with and without adding steroid. Cells containing the peptide were stained with a dye (Sytox) capable of detecting cells that have a compromised membrane (i.e. dead/dying cells). The plates were analyzed on a CCD imaging system customized from a PixelVision Spectra Video™ Series imaging camera (1100×330 back-illuminated array, Pixel Vision, Tigurd, Oreg.), Pixel Vision PixelView™ 3.03 software, two 50 mm/f2 Olympus macro focusing lens mounted front to front, four 20750 Fostec xenon light sources (Schott-Fostec, Auburn, N.Y.), four 8589 Fostec light lines, a 4457 Daedal stage, and supporting mechanical fixtures. Following these procedures, saponin was added to each well to permeabilize the remaining cells prior to recording an other CCD image to determine total cell number. The number of dead and live cells in each well was compared with the appropriate controls to assess if any cytotoxic/cytostatic properties were associated with the peptides under study. Furthermore, the cyto-inhibitory nature of each peptide was described by its “kill index”, the normalized ratio of the dead cell number over the total cell number.

FACs Analysis

Cells were transduced in 96-well plate format as described above. However, instead of performing the sytox-cytotoxicity assay, cells were prepared for FACs analysis by removing them from the plate by trypsinization (0.05% for 5′, Gibco) and FACs analysis was performed using a Coulter analyzer (EPICS XL-MCL, EXPO software, Build 320, excitation 488 nM argon laser, emission FL1=525BP+/−10 nm run at 15 milliwatts).

Promoters: pADH, pGPD, GAL 1, CUP1

Activators: VP16, GALAC, B42

LBD: ER^{LBD}, PR^{LBD},AR^{LBD}

Mammalian promoters: HIV2, CMV, RSV

Tables 2 and 3 demonstrate constructs and yeast strains that were either utilized or can be utilized in the compositions and methods described herein.

TABLE 2
Plasmids

| Plasmid | Markers | Reference |
|---|---|---|
| TSB390 | pADH-GAL^{BD}: | This study; this vector is a control |
| | INF5, CEN, TRP1, | without the PR^{LBD}. |
| | Kan^{R} | |
| TSB400/ | pADH-GAL^{AD}- | This vector contains a fragment |
| pBal26 | IN(1-210), CEN, | of integrase that interacts |
| | LEU2, Amp^{R} | with INF5 peptide |
| TSB407 | pADH-GAL^{BD}: PR- | This study; this vector is used for |
| | LBD: INF5, CEN, | control release of Peptide using |
| | TRP1, Kan^{R} | progesterone |
| TSB408 | pADH-GAL^{BD}: PR- | This study; same |
| | LBD: INF5, CEN, | |
| | TRP1, Kan^{R} | |
| TSB410 | pADH-GAL^{BD}: PR- | This study; this vector is a |
| | LBD: GAL^{AD}, CEN, | positive control. This tripartite |
| | TRP1, Kan^{R} | construct is activated in the |
| | | presence of progesterone. A CEN |
| | | vector reduces variability due |
| | | to plasmid copy number in yeast. |
| TSB413 | pADH-GAL^{AD}- | This study; This vector contains a |
| | IN(1-210), CEN, | fragment of Integrase that interacts |
| | LEU2, Kan^{R} | with INF5 peptide. A CEN vector |
| | | reduces variability due to plasmid |
| | | copy number in yeast. |
| TSB428 | pADH-GAL^{BD}: PR- | This study; this vector is used to |
| | LBD: Stuffer, CEN, | clone in inserts and libraries. |
| | TRP1, Kan^{R} | The stuffer fragment reduces |
| | | background in cloning. |
| TSB431 | pADH-VP16-IN(1- | This vector is used to increase signal |
| | 210), CEN, LEU2, | strength inserts and libraries. VP16 |
| | Kan^{R} | is a stronger activator than GAL4^{AD} |
| pVT702 | pADH-GAL^{AD}, 2u, | Base vector |
| | LEU2, Amp^{R} | |
| pVT2114 | pADH-GAL^{BD}, | Base vector |
| | CEN, TRP1, Kan^{R} | |
| pVT3210 | pADH-GAL^{AD}, | Base vector |
| | CEN, LEU2, Kan^{R} | |
| pVT3223 | pADH-GAL^{AD}-pepA | Control peptide vector |
| | CEN, LEU2, Kan^{R} | |

TABLE 3
Yeast Strains Used in this Study

| Strain | Markers |
|---|---|
| TSY200 | TSY 201 MATa ura3-52, his3-200, ade2-201, lys2-801, trp1- |
| | 901, leu2-3,112, gal4-542, gal80-538 LYS2::GAL2_{UAS}- |
| | GAL1_{TATA}-HIS3, URA3::GAL4_{17mers(X3)-} CyC1_{TATA}-lacZ. |
| TSY201 | TSY 201 MATa ura3-52, his3-200, ade2-201, lys2-801, trp1- |
| | 901, pdr5delta, sng2delta, leu2-3,112, gal4-542, gal80-538 |
| | LYS2::GAL2_{UAS}-GAL1_{TATA}-HIS3, URA3::GAL4_{17mers(X3)-} |
| | CyC1_{TATA}-lacZ. |
| TSY355 | TSY 201 MATa ura3-52, his3-200, ade2-201, lys2-801, trp1- |
| | 901, pdr5delta, sng2delta, leu2-3,112, gal4-542, gal80-538 |
| | LYS2::GAL2_{UAS}-GAL1_{TATA}-HIS3, URA3::GAL4_{17mers(X3)-} |
| | CyC1_{TATA}-lacZ. [pADH-GAL-DBD: PR-LBD: INF5, CEN, |
| | TRP1, Kan^{R}] |
| TSY356 | TSY 201 MATa ura3-52, his3-200, ade2-201, lys2-801, trp1- |
| | 901, pdr5delta, sng2delta, leu2-3,112, gal4-542, gal80-538 |
| | LYS2::GAL2_{UAS}-GAL1_{TATA}-HIS3, URA3::GAL4_{17mers(X3)-} |
| | CyC1_{TATA}-lacZ. |
| TSY359 | TSY 201 MATa ura3-52, his3-200, ade2-201, lys2-801, trp1- |
| | 901, pdr5delta, sng2delta, leu2-3,112, gal4-542, gal80-538 |
| | LYS2::GAL2_{UAS}-GAL1_{TATA}-HIS3, URA3::GAL4_{17mers(X3)-} |
| | CyC1_{TATA}-lacZ. |
| TSY361 | TSY 201 MATa ura3-52, his3-200, ade2-201, lys2-801, trp1- |
| | 901, pdr5delta, sng2delta, leu2-3,112, gal4-542, gal80-538 |
| | LYS2::GAL2_{UAS}-GAL1_{TATA}-HIS3, URA3::GAL4_{17mers(X3)-} |
| | CyC1_{TATA}-lacZ. |
| | MATa ura3-52, his3-200, ade2-201, lys2-801, trp1-901, leu2- |
| | 3,112, gal4-542, gal80-538 LYS2::GAL2_{UAS}-GAL1_{TATA}-HIS3, |
| | URA3::GAL4_{17mers(X3)-} CyC1_{TATA}-lacZ. |
| | MATa ura3-52, his3-200, ade2-201, lys2-801, trp1-901, leu2- |
| | 3,112, gal4-542, gal80-538 LYS2::GAL2_{UAS}-GAL1_{TATA}-HIS3, |
| | ura3::GAL4_{17mers(X3)-}CyC1_{TATA}-lacZ. |
| | MATa ura3-52, his3-200, ade2-201, lys2-801, trp1-901, leu2- |
| | 3,112, gal4-542, gal80-538 LYS2::GAL2_{UAS}-GAL1_{TATA}-HIS3, |
| | ura3::GAL4_{17mers(X3)-} CyC1_{TATA}-GFP. |
| | Mat a, trp1-901, leu2-3,112, ura3-52, his3-200, gal4 Δ, |
| | gal 80Δ, LYS2::GAL1_{UAS}-GAL1_{TATA}-HIS3, GAL2_{UAS}- |
| | GAL2_{TATA}-ADE2, URA3: MEL1_{UAS}-MEL1_{TATA}-lacZ)[[pADH- |
| | GAL-DBD: PR-LBD: Bait, CEN, TRP1, Kan^{R}] |
| | Mat alpha, trp1-901, leu2-3,112, ura3-52, his3-200, |
| | gal4 Δ, gal 80Δ, LYS2::GAL1_{UAS}-GAL1_{TATA}-HIS3, |
| | GAL2_{UAS}-GAL2_{TATA}-ADE2, URA3: MEL1_{UAS}-MEL1_{TATA}- |
| | lacZ) [pADH-VP16-randompep, 2u, LEU2, Amp^{R}] |
| | Mat a, trp1-901, leu2-3,112, ura3-52, his3-200, gal4 Δ, |
| | gal 80Δ, LYS2::GAL1_{UAS}-GAL1_{TATA}-HIS3, GAL2_{UAS}- |
| | GAL2_{TATA}-ADE2, URA3: MEL1_{UAS}-MEL1_{TATA}-lacZ)[[pADH- |
| | GAL-DBD: PR-LBD: randompep, CEN, TRP1, Kan^{R}] |
| | Mat alpha, trp1-901, leu2-3,112, ura3-52, his3-200, |
| | gal4 Δ, gal 80Δ, LYS2::GAL1_{UAS}-GAL1_{TATA}-HIS3, |
| | GAL2_{UAS}-GAL2_{TATA}-ADE2, URA3: MEL1_{UAS}-MEL1_{TATA}- |
| | lacZ) [pADH-GALAC-Bait, CEN, LEU2, Amp^{R}] |

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

1. A kit comprising:
(a) a first polynucleotide comprising a nucleic acid sequence encoding a first hybrid protein, wherein the first hybrid protein comprises a DNA binding domain (DBD) that binds an expression control sequence operably linked to a detectable reporter gene, wherein the first hybrid proteins further comprise a first heterologous interacting domain or the nucleic acid sequence encoding the first hybrid protein further comprises a cloning site for inserting a first heterologous interacting domain in frame with the DBD; and
(b) a second polynucleotide comprising a nucleic acid sequence encoding a second hybrid protein, wherein the second hybrid protein comprises at least an activation domain (AD) that activates transcription of the detectable reporter gene when associated with the DBD bound to the expression control sequence, wherein the second hybrid protein further comprise a second heterologous interacting domain or the nucleic acid sequence encoding the second hybrid protein further comprises a cloning site for inserting a second heterologous interacting domain in frame with the AD;
wherein at least one of the first or second hybrid proteins further comprises a ligand binding domain (LBD) of a nuclear receptor,
further comprising a regulatory element that binds to the LBD and prevents an interaction between the first hybrid protein and the second hybrid protein.

2. (canceled)

3. The kit of claim 1, wherein the regulatory element is a heat shock protein (Hsp).

4. The kit of claim 3, wherein the regulatory element is Hsp90.

5. The kit of claim 1, further comprising a ligand capable of displacing the regulatory element, wherein displacement of the regulatory element allows the first hybrid protein to interact with the second hybrid protein and to activate the detectable reporter gene.

6. The kit of claim 5, wherein the ligand is a steroid that binds the nuclear receptor with greater affinity that the regulatory element.

7. The kit of claim 6, wherein the nuclear receptor comprises estrogen receptor, androgen receptor, progesterone receptor, testosterone receptor, or glucocorticoid receptor.

8. The kit of claim 7, wherein the steroid is selected from the group consisting of Cortisol, hydrocortisone, estrogen, estradiol, estrone, progesterone, testosterone, and a combination thereof.

9. The kit of claim 1, further comprising an agent that inhibits or prevents the interaction between the first hybrid protein and the second hybrid protein.

10. The kit of claim 1, wherein the reporter gene encodes β-galactosidase, green fluorescent protein, luciferase, β-glucuronidase, chloramphenicol acetyltransferase, or alkaline phosphatases; wherein the reporter gene comprises URA3, LYS2, ADE2, ADE3, HIS3, or TRP1; or any combination thereof.

11. The kit of claim 1 further comprising a host cell comprising the detectable reporter gene.

12. The kit of claim 11, wherein the host cell comprises a cultured eukaryotic cell.

13. The kit of claim 12, wherein the host cell comprises Saccharomyces cerevisae, wherein the detectable reporter gene comprises LacZ.

14. The kit of claim 13, wherein the first hybrid protein has the formula:
GAL^{BD}-LBD-B,
wherein the second hybrid protein has the formula:
GAL^{AD}-P,
wherein GAL^{BD} is the DNA binding domain of Gal4,
wherein GAL^{AD} is the activation domain of Gal4,
wherein LBD is the ligand binding domain of a nuclear receptor that binds a heat shock protein and a steroid hormone, and
wherein B and P are heterologous peptide sequences.

15. A controlled release hybrid system comprising a host cell, the cell comprising:
(a) a detectable reporter-gene operably linked to an expression control sequenced;
(b) a first hybrid protein containing at least a DNA-binding domain (DBD) that binds the expression control sequence, and a second hybrid protein comprising at least an activation domain (AD) of a transcriptional activator, wherein interaction between the first hybrid protein and the second hybrid protein results in transcriptional activation of the detectable reporter-gene, wherein at least one or more of the first hybrid protein and the second hybrid protein further comprise a ligand binding domain (LBD);
(c) a regulatory element that binds to the LBD and prevents an interaction between the first hybrid protein and the second hybrid protein; and
(d) a ligand capable of displacing the regulatory element, wherein displacement of the regulatory element allows the first hybrid protein to interact with the second hybrid protein and to activate the detectable reporter-gene.

16. (canceled)

17. The controlled release hybrid system of claim 15, further comprising an agent that inhibits or prevents the first hybrid protein from interacting with the second hybrid protein.

18. The controlled release hybrid system of claim 15, wherein the first hybrid protein comprises at least a DBD and a first heterologous interacting domain and wherein the second hybrid protein comprises at least a second heterologous interacting domain and an AD.

19. The controlled release hybrid system of claim 15, wherein the first hybrid protein comprises a DBD, a LBD, and a first heterologous interacting domain.

20. The controlled release hybrid system of claim 15, wherein the second hybrid protein comprises a second heterologous interacting domain, a LBD, and an AD.

21. The controlled release hybrid system of claim 15, wherein the LBD is a Nuclear Receptor LBD, wherein the regulatory element is a heat shock protein (Hsp), and wherein the ligand is a steroid that binds the LBD and displaces Hsp from the LBD.

22. The controlled release hybrid system of claim 21, wherein the steroid comprises a gonane derivative, progestin, androgen, corticosteroid, anabolic steroid, or a combination thereof.

23. The controlled release hybrid system of claim 21, wherein the steroid is selected from the group consisting of Cortisol, hydrocortisone, estrogen, estradiol, estrone, progesterone, testosterone, and a combination thereof.

24. The controlled release hybrid system of claim 15, wherein the detectable reporter gene encodes beta-galactosidase, green fluorescent protein, luciferase, beta-glucuronidase, chloramphenicol acetyltransferase, alkaline phosphatase, or any combination thereof.

25. The controlled release hybrid system of claim 15, wherein the detectable reporter gene comprises URA3, LYS2, ADE2, ADE3, HIS3, TRP1, or any combination thereof.

26. The controlled release hybrid system of claim 15, wherein the LBD comprises an estrogen receptor (ER) LBD, and androgen receptor (AR) LBD, a progesterone receptor (PR) LBD, a testosterone receptor (TR) LBD, a glucocorticoid receptor (GR) LBD, or any combination thereof.

27. The controlled release hybrid system of claim 15, wherein the cultured host cell comprises a eukaryotic cell.

28. The controlled release hybrid system of claim 27, wherein the cultured host cell comprises Saccharomyces cerevisae,

29. The controlled release hybrid system of claim 15, wherein the detectable reporter gene comprises LACZ, wherein the first hybrid protein comprises the DBD of Gal4, and wherein the second hybrid protein comprises the AD of Gal4.

30. A method for identifying agents that reduces or prevents intermolecular binding between a first interacting peptide and a second interacting peptide comprising:
(a) administering to a host cell containing a detectable reporter gene operably linked to an expression control sequence:
(i) a first polynucleotide comprising a nucleic acid sequence encoding a first hybrid protein, wherein the first hybrid protein comprises a DNA binding domain (DBD) that binds the expression control sequence, wherein the first hybrid proteins further comprise the first interacting peptide; and
(ii) a second polynucleotide comprising a nucleic acid sequence encoding a second hybrid protein, wherein the second hybrid protein comprises at least an activation domain (AD) that activates transcription of the detectable reporter gene when associated with the DBD bound to the expression control sequence, wherein the second hybrid protein further comprise the second interacting peptide;
wherein at least one of the first or second hybrid proteins further comprises a ligand binding domain (LBD) of a nuclear receptor;
(b) administering to the host cell a regulatory agent that binds the LBD and prevents an interaction between the first hybrid protein and the second hybrid protein;
(c) administering to the host cell a ligand that is capable of displacing the regulatory element, wherein displacing the regulatory element allows the first hybrid protein to interact with the second hybrid protein;
(d) comparing expression of the detectable reporter gene in the host cell of step (c) to a control, wherein a reduction in gene expression compared to a control is an indication that the agent inhibited the interaction between the first interacting peptide and the second interacting peptide.

31. A method of identifying a mutation in a target protein that gives rise to drug resistance and identifying a non-resistant therapeutic agent, comprising
(a) providing an inhibitory peptide that binds the target protein;
(b) screening target protein mutants for a target protein variant with reduced binding to the inhibitory peptide;
(c) screening a library of candidate peptides for a peptide variant that binds the target protein variant identified in step (b) but does not bind the target protein; and
(d) screening a chemical library for an agent that inhibits the interaction between the target protein variant identified in step (b) and the peptide variant identified in step (c).

32. The method of claim 31, wherein the inhibitory peptide of step (a) is identified by a yeast-two hybrid assay detecting the interaction between a candidate peptide and the target protein.

33. The method of claim 31, wherein the screening of step (b) comprises a yeast-two hybrid assay detecting the interaction between the inhibitory peptide of step (a) and a target protein mutant

34. The method of claim 31, wherein the screening of step (c) comprises a first yeast-two hybrid assay detecting the interaction between a candidate peptide and the target protein, and a second yeast-two hybrid assay detecting the interaction between the candidate peptide and the target protein variant identified in step (b).

35. The method of claim 31, wherein the screening of step (d) comprises a yeast-two hybrid assay detecting the ability of a candidate agent to inhibit the interaction between the target protein variant identified in step (b) and the peptide variant identified in step (c).

36. The method of claim 31, wherein the target protein is HIV integrase.

37. The method of claim 36, wherein the inhibitory peptide of step (a) comprises the amino acid sequence SEQ ID NO:7.

38. The method of claim 31, wherein the target protein mutants of step (b) are naturally occurring.

39. The method of claim 31, wherein the target protein mutants of step (b) are produced by directed mutagenesis.

40. The method of claim 31, wherein the candidate peptides have at least 90% sequence identity to the inhibitory peptide of step (a).


SEQUENCE LISTING (19 sequences)

SEQ ID NO: 1 - DNA, 468 bp, Artificial Sequence
  Synthetic Construct
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggtattgcaatacccagctttgact

SEQ ID NO: 2 - protein, 156 aa, Artificial Sequence
  GAL- DNA BINDING DOMAIN
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSPVLQYPALT

SEQ ID NO: 3 - DNA, 483 bp, Artificial Sequence
  GAL-ACTIVATION DOMAIN
atggataaagcggaattaattcccgagcctccaaaaaagaagagaaaggtcgaattgggtaccgccgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagatctttaatacgactcactatagggcgagcgccgccatggagtacccatacgacgtaccagattacgctcatatg

SEQ ID NO: 4 - protein, 159 aa, Artificial Sequence
  GAL-ACTIVATION DOMAIN
MDKAELIPEPPKKKRKVELGTAANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKEIFNTTHYRASAAMEYPYDVPDYA

SEQ ID NO: 5 - DNA, 762 bp, Artificial Sequence
  Progesterone Ligand Binding Domain
atgatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacatgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaagcc

SEQ ID NO: 6 - protein, 254 aa, Artificial Sequence
  PROGESTERONE LIGAND BINDING DOMAIN
MIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAAQLPKILAGMVKPLLFHKA

SEQ ID NO: 7 - protein, 15 aa, Artificial Sequence
  HIV INTEGRASE BINDING PEPTIDE
LYETILILLFLDVDT

SEQ ID NO: 8 - DNA, 45 bp, Artificial Sequence
  HIV INTEGRASE BINDING PEPTIDE
ttgtacgagactatcttgattttgctgtttcttgacgtggatacg

SEQ ID NO: 9 - DNA, 644 bp, Artificial Sequence
  HIV INTEGRASE
actttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatacagacaatggcagcaatttcaccagtgctacggttaaggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactagctctagctag

SEQ ID NO: 10 - protein, 210 aa, Artificial Sequence
  HIV INTEGRASE
FLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTSATVKAACWWAGIKQEFGIPYNPQSQGVVESMNKELKKIIGQVRDQAEHLKTAVQMAVFIHNFKRKGGIGGYSAGERIVDIIATDIQT

SEQ ID NO: 11 - DNA, 897 bp, Artificial Sequence
  Full Length HIV Integrase
tttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggtaaggatccatcgagctcgagctgcagatgaatcg

SEQ ID NO: 12 - protein, 299 aa, Artificial Sequence
  Full Length HIV Integrase
FLDGIDKAQEEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDIQTKELQKQITKIQNFRVYYRDSRDPVWKGPAKLLWKGEGAVVIQDNSDIKVVPRRKAKIIRDYGKQMAGDDCVASRQDEVRIHRARAADES

SEQ ID NO: 13 - DNA, 756 bp, Artificial Sequence
  Androgen Receptor Ligand Binding Domain
atgtgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacaccgcg

SEQ ID NO: 14 - protein, 251 aa, Artificial Sequence
  Androgen Receptor Ligand Binding Domain
CQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTA

SEQ ID NO: 15 - DNA, 921 bp, Artificial Sequence
  Estrogen Receptor Ligand Binding Domain
atgcctatcgatatcagtcgagcttctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgatgatcacggcg

SEQ ID NO: 16 - protein, 306 aa, Artificial Sequence
  Estrogen Receptor Ligand Binding Domain
PIDISRASAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSMITA

SEQ ID NO: 17 - DNA, 53 bp, Artificial Sequence
  Synthetic Construct
gctttgactcatatgatacagttgattccaccactgatcaacctgttaatgag

SEQ ID NO: 18 - DNA, 47 bp, Artificial Sequence
  Synthetic Construct
cctcggcccatatgggctttatgaaagagaaggggtttcaccatccc

SEQ ID NO: 19 - DNA, 13 bp, Artificial Sequence
  Synthetic Construct
cggccacgctgga